(19) United States
(12) Patent Application Publication
(10) Pub. No.: US 2026/0048999 A1
(43) Pub. Date: Feb. 19, 2026

(54) ONE-DIMENSIONAL LEPIDOCROCITE COMPOSITIONS

(71) Applicants: DREXEL UNIVERSITY, Philadelphia, PA (US); MURATA MANUFACTURING CO., LTD., Kyoto (JP)

(72) Inventors: Michel W. BARSOUM, Moorestown, NJ (US); Hussein Osama BADR, Philadelphia, PA (US); Masashi KOYANAGI, Kyoto (JP); Akimaro YANAGIMACHI, Kyoto (JP)

(21) Appl. No.: 19/103,604

(22) PCT Filed: Aug. 17, 2023
(86) PCT No.: PCT/US2023/072374

§ 371 Date: Feb. 13, 2025

Related U.S. Application Data
(60) Provisional application No. 63/398,782, filed on Aug. 17, 2022.
(60) Provisional application No. 63/373,490, filed on Aug. 25, 2022.

(51) Int. Cl. C01G 23/053; A61K 9/70; A61K 33/22; B82Y 5/00; B82Y 30/00; B82Y 40/00; C08K 7/08; H01M 4/48

(52) U.S. Cl. CPC C01G 23/053; A61K 9/70; A61K 33/22; C08K 7/08; H01M 4/48; B82Y 5/00; B82Y 30/00; B82Y 40/00; C01P 2002/72; C01P 2002/77; C01P 2002/82; C01P 2004/03; C01P 2004/04; C01P 2004/16; C01P 2004/24; C08K 2201/004; C08K 2201/011

(57) ABSTRACT

Provided are methods to convert—through a bottom-up approach—binary and ternary titanium carbides, nitrides, borides, phosphides, aluminides, and silicides into lepidocrocitic nanofilaments that in some cases self-assemble into 2D flakes by immersing them in a quaternary ammonium solution at moderate temperatures. The resulting flakes can be C-containing layers that are in turn comprised of nanofilaments in cross-section, some of which nanofilaments can be few microns long in some instances.

### CROSS REFERENCE TO RELATED APPLICATIONS

The present application claims priority to and the benefit of U.S. patent application No. 63/398,782 (filed Aug. 17, 2022) and of U.S. patent application No. 63/373,490 (filed Aug. 25, 2022). All foregoing applications are incorporated herein by reference in their entireties for any and all purposes.

### TECHNICAL FIELD

The present disclosure relates to the field of 1D and 2D materials and to the field of metal oxide-based nanomaterials.

### BACKGROUND

Nanostructured, (NS) titanium dioxides, TiO₂, have been, and remain, of significant research interest due to their unique physical and chemical properties as well as their potential application in a wide range of fields including paint pigment, catalysis, photocatalysis, photoluminescence, gas sensors, solar and fuel cells among many others.¹⁻¹¹ One dimensional, 1D, and two-dimensional, 2D, materials possess characteristics and properties that their three-dimensional, 3D, solids do not. Arguably the most important difference is in their much higher surface areas. In terms of properties, low-dimensional solids allow for quantum confinement and more active catalytic sites. Accordingly, there is a long-felt need in the art for 1D materials having useful characteristics.

### SUMMARY

A bottom-up, sol-gel based, one-pot, inexpensive and highly scalable process can be used to make TiO₂-based, 1D nanofilaments, NFs.¹² In our method, we simply immerse earth abundant, non-toxic, water insoluble binary and ternary titanium carbides, nitrides, borides, etc., in tetramethylammonium hydroxide, TMAH, aqueous solutions at temperatures in the 50 to 85° C. range for tens of hours under ambient pressures. This procedure converts the precursors to 1D NFs, that in turn self-assemble into quasi 2D flakes upon washing with water and filtering.¹² We show that the structure of our 1D NFs is lepidocrocite-based, henceforth referred to as 1DL. We also show that the NFs grow along the a, or the 200 direction and stack along both b- and c-directions.

### BRIEF DESCRIPTION OF THE FIGURES

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1: a) XRD patterns of 2 samples one washed with ethanol; the other washed with ethanol and then 0.5 M LiCl (see Methods Section). Positions of the (200) and (002) peaks at 2 □≈48° and ≈62° are crystallographic and not processing invariant. The positions of all other peaks are. Note log scale on y-axis. Yellow bands outline the three arcs/rings observed previously in SAD patterns of 2D flakes in TEM.¹² b) Schematic of DFT generated structure with TiO₂-ribbons stacked normal to b-axis. Also traced are all non-basal planes predicted. Rectangle on bottom right denotes a unit cell with lattice parameters a and b. Note while a is crystallographic, b is not and depends on spacing between ribbons here chosen to 7.5 Å. The same is true of stacking along 001. c) Schematic of (001) plane assuming it is 2 Ti atoms wide. Spacing between 2 adjacent Ti atoms, along c, or d002 is ≈1.5 Å, which gives rise to peak ≈62° 2□. seen in diffraction and SAD pattern.¹² Also shown in b and c are the approximate “thicknesses”—measured from outermost O to outermost O—of 2-atom thick Ti ribbons.

FIG. 2: Raman spectra of 6 samples that were washed differently (see Methods Section). In all cases the resulting spectra were consistent with those of lepidocrocite. Inset shows effect of laser power on spectra. At high power, the material transforms from lepidocrocite to anatase. The 10, 50 and 100% laser powers correspond to 6, 29 and 52 mWcm⁻², respectively.

FIG. 3: ABF TEM micrograph of a bundle of individual 1DL NFs oriented along the fiber axis. Lower left inset is FFT of region outlined by blue square. Superimposed on the FFT, as red circles, are the indices predicted after the spacing of layers along b was chosen to be 7.5 Å. Agreement is excellent. Lower right inset shows schematic of lepidocrocite layers (not

to scale) stacked along the b-axis. Growth direction is along [200], which coincides with bundle axis. Inset top left is SAD pattern generated by software assuming distance between ribbons along b is 7.5 Å. Planes outlined in FIG. 1b are denoted by red arrow. Inset top right is HAADF image of region shown in figure. Precursor was TiB₂, reacted in TMAH for 5 days at 80° C.

FIG. 4: Low angle annular dark field TEM micrograph of same sample as shown in FIG. 3 at, a) low magnification, and b) higher magnification of region enclosed in green square in a. Scale bar in 5 nm. Zig-zag nature of Ti atoms in the nanoribbons in area enclosed by red circle is obvious. Their 2-layered nature is also.

FIG. 5: ABF TEM micrograph of same sample as in FIG. 3, but focusing on different regions. Top inset is FFT produced from blue square showing diffuse rings with no clear diffraction spots. Lower inset is FFT taken from green square showing clear diffraction spots indicating the region is largely crystalline.

FIG. 6: LAADF TEM image of a loose NF bundle derived from TiC; b) Magnified view of dashed regions in a shows crystalline contrast the NFs. A region of well-ordered NFs shows atomic column contrast consistent lepidocrocite oriented along [100].

FIG. 7: Tauc plots as a function of various washing procedures. (see Methods Section).

FIG. 8: Scalable synthesis of nanofilaments-based mesoporous particles. Schematic of (a) Temperature-controlled shaking incubator used to convert TiB2 precursor powder into nanofilaments-based mesoporous particles. (b) Washing protocol followed to remove any unreacted TMAH salt. (c) DFT-generated lepidocrocite structure showing 2 Ti atoms-thick ribbons growing along [100] and stacking along [010] (viz., a and b crystallographic directions, respectively). (d) Various cations (both mono and divalent ones) intercalation in the interfilamentous gallery.

FIG. 9: (a) XRD patterns—on log scale—of TiB2 precursor powder (top black curve), as well as samples reacted for 1 d to 5 d then washed with ethanol before they were dried at 50° C. in air overnight. The peaks at ≈26°, 48° and 62° θ0 correspond to 110, 200 and 002 planes of lepidocrocite, respectively. These values correspond 3 arcs/rings observed in SAD patterns of 1DLs in TEM. The 0k0 peaks are denoted by asterisks. Dashed black lines denote TiB2 diffraction peaks. (b)-(d) SEM micrographs—at various magnifications—of mesoporous particles after 5 d of reaction. Inset in (b) shows MPPs size distribution obtained using ImageJ from the micrograph. FIGS. 15-17 show more SEM images.

FIG. 10: (a) STEM imaging of mesoscopic particle from TiB2-derived sample shook in TMAH at 80° C. for 5 d, washed with ethanol, then dehydrated at 50° C. overnight in open air. (b) and (c) LAADF STEM micrographs—at various magnification—of bundles of 1DL nanofilaments oriented along the fiber axis. Inset in (b) shows FFT of produced from area bounded by yellow square. (d)-(h) EDX elemental map of mesoscopic particle shown in inset in (a).

FIG. 11: Characterization of mesoporous particles, (a)-(c) XRD patterns and (d)-(k) SEM micrographs, prepared by shaking TiB2 precursor powder in TMAH solution at 80° C. for 5 d, washing with solvent/solutions labeled on the panels, then letting to dry at 50° C. in open air.

FIG. 12: (a) ζ-Potential (left y-axis) and average hydrodynamic size (right y-axis) of TiB2-derived samples shook in TMAH at 80° C. for 5 d then washed with solvents/solutions labeled on panel.

FIG. 13: SEM micrographs, at various magnifications, of samples after reaction for (a,b,c) 1 day, and (d,e,f) 5 days. Samples were reacted in TMAH at 80° C. then washed with ethanol before drying at 50° C. in ambient air. Micrographs after reaction for 2, 3 and 4 d are shown in FIG. 25 and are no different than the ones shown here.

FIG. 14: Location of reaction and morphology. a) NFs form at the solid/liquid interface by the formation of TiO₆ octahedra and their attachment to the bottom of the growing NF. b) Effect of washing procedures on final morphology.

FIG. 15: SEM micrographs, at various magnifications, of samples washed with ethanol, then dried overnight in open air at 50° C. All samples were reacted for 5 d at 80° C. in shaker.

FIG. 16: SEM micrographs, at various magnifications, of samples washed with ethanol, then a LiCl 0.5 M aqueous solution and lastly water, before allowing them to dry in open air at 50° C. overnight. All samples were reacted for 5 d at 80° C. in shaker.

FIG. 17: SEM micrographs, at different magnifications, of samples washed with ethanol, then immersed in 5M NaCl aqueous solution, and water, before allowing them to dry in open air at 50° C. overnight. All samples were reacted for 5 d at 80° C. in shaker.

FIG. 18: HAADF imaging and EELS elemental mapping of MPPs obtained after ethanol washing. (a) HAADF imaging of bundles of NFs from which EELS maps were acquired. (b) HAADF image acquired simultaneously with the element maps. No observable changes in the morphology were observed in subsequent scans. (c) Elemental composition collected from area outlined with dashed box in (b) and were calculated using a Hartree-Slater model. (d)-(g) EELS elemental maps of Ti, O, C, and N respectively.

FIG. 19: (a) XRD patterns of samples before and after ion exchange with salt aqueous solutions labeled on panels. (b)-(c) same as (a) but after ethanol washing the powders were further treated in LiCl aqueous solution, then stirred in aqueous solutions of salts labeled on panels. Vertical dashed blue line aligned—across panels—at ˜9.5 Å designating d-spacing of the LiCl washed sample. Vertical dashed black lines/grey band designate the lowest and highest d-spacing values for the 110 non-basal reflection with respect to various intercalants between the NFs. Vertical red dashed lines/bands refer to the 200 and 002 lepidocrocite reflections at 2θ values of ˜48° and 62°, respectively. Asterisks denote peaks of unreacted TiB2that we use as internal standard to align the XRD patterns.

FIG. 20: Characterization of mesoporous particles, (a) XRD patterns and (b) SEM micrograph, prepared by shaking TiB2 precursor powder in TMAH solution at 80° C. for 5 d, washed with ethanol till neutralization, stirred directly in solutions labeled on the panels, then let to dry at 50° C. in open air. Note log scale on the y-axis.

FIG. 21: (a) and (b) Still frames of ethanol washed powders that are dispersed in ethanol and water, respectively. (c) Same as (a) but for powders that were treated in LiCl aqueous solution then dispersed in water.

FIG. 22. (a) and (b) AFM scans of colloidal suspensions (obtained by heating TiC in TMAH at 80° C. for 3 d, washed with ethanol till neutralization, then dispersed in water before and after dilution 500×, respectively, then drop casting on a glass slide. Inset shows height profile corresponding to blue line in (d); thinnest filaments are 1.5 nm high. The figure is reproduced from Mat. Today, Elsevier with permission (license number 5591960829133).F

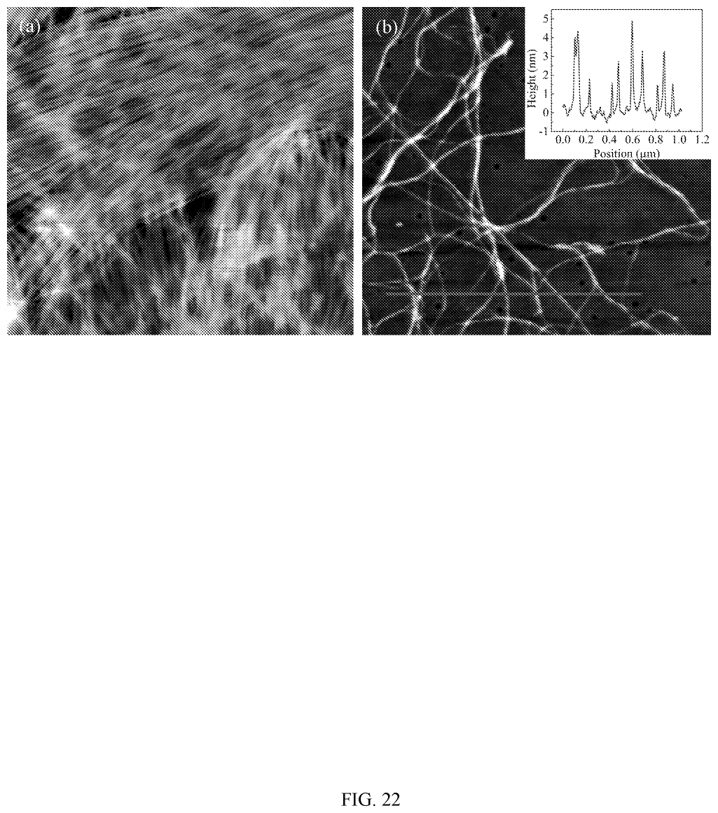

FIG. 23. (a) Thermogravimetric plots for MPPs prepared by shaking TiB2 in TMAH solution at 80° C. for 5 d, then washed with ethanol till neutralization. Some samples were further treated in LiCl or NaCl solution then rinsed with water. All powders were let to dry at 50° C. in open air. Vertical dashed lines are guides to 200° C. and 400° C. (b)-(c) XRD patterns for MPPs processed with conditions labeled on the panels. All TGA powders ramped at 10° C./min to 800° C. in Ar. Black and blue asterisks in (b) and (c) denote anatase and rutile, respectively, obtained after TGA. Vertical arrows on middle green line in (c) denotes Li2Ti2O4, while those on bottom red line denotes Na₂Ti₆O₁₃ obtained after TGA.

FIG. 24: (a)-(c) SEM micrographs of TiB2-derived mesoporous particles (washed with ethanol and dried at 50° C. in open air) heated under Ar up to 200° C. (denoted by red curve in FIG. 23b). (c)-(f) Same as (a)-(c) but for powders heated up to 800° C. (denoted by green curve in FIG. 23b).

FIG. 25: SEM micrographs, at various magnifications, of samples after reaction for (a,b,c) 2 day, (d,e,f) 3 days, and (g,h,i) 4 days. Samples were reacted in TMAH at 80° C. then washed with ethanol before drying at 50° C. in ambient air.

FIG. 45: SEM micrographs revealing TiB2 particles transforming into 1DL NFs via localized corrosion. Samples were reacted in TMAH at 80° C. for 3d then washed with ethanol before drying at 50° C. in ambient air.

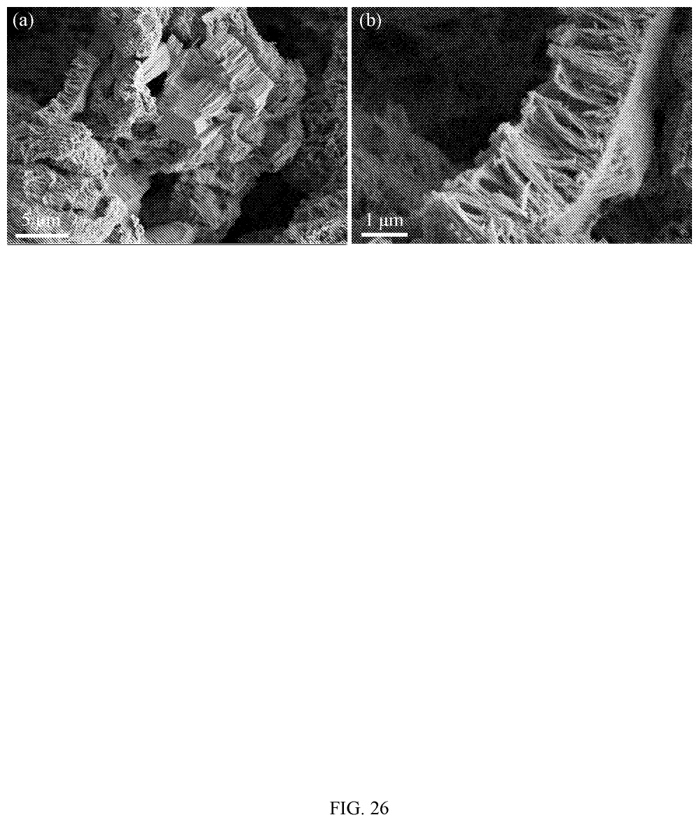

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

### Results and Discussion

All experimental details can be found in Methods Section.

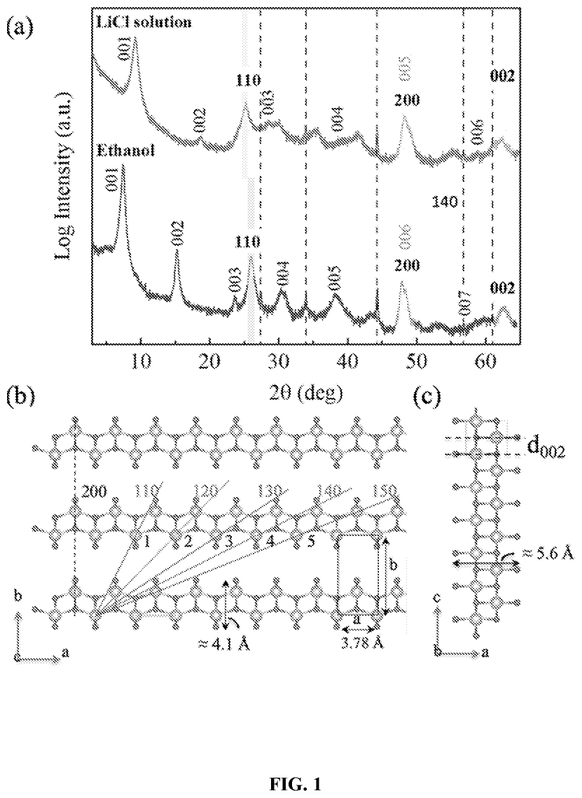

FIG. 1 plots XRD patterns—on log scale—for 2 samples that were synthesized by reacting TiB₂ powders with TMAH at 80° C. for 5 days. After reaction, the resulting powders were washed with ethanol until the pH was ˜ 7. In one case, the powders were dehydrated straight from ethanol at 50° C. in open air (bottom blue curve in Fig. la). In the other case, sediments were further stirred in a LiCl solution then rinsed with DI water before allowing the powders to, again, naturally dry in open air (top red curve in FIG. 1a).

Vertical dashed lines in FIG. 1 designate two low-intensity unreacted TiB₂ peaks that were used as internal standards. When the powders were washed with ethanol, the XRD patterns were characterized by 7 basal reflections with a d-spacing of ≈11.5 Å, due to the stacking of “2D” flakes comprised of in-plane alignment of 1DL (see below). After washing with LiCl (see Methods Section), the d-spacing value dropped to ≈9.5 Å confirming the replacement of TMA″ cations with Li⁺. The yellow bands in FIG. 1a denote lepidocrocite non-basal reflections, at 2□ values of ≈26°, ≈48° and ≈62° 2□. These peak positions are in excellent agreement with our previous XRD patterns, as well as with rings previously observed in SAD patterns in TEM.¹²

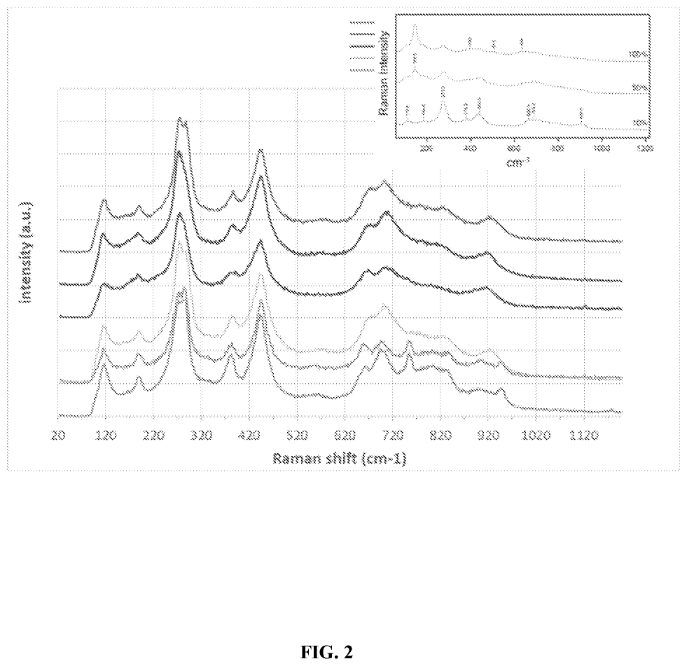

FIG. 2 presents the Raman spectra of 6 samples processed in different ways outlined in Methods Section. In all cases, the spectra obtained were consistent with lepidocrocite.¹³ In retrospect, it is now clear that the laser power used to obtain our previous spectra,¹² was too high which resulted in a lepidocrocite-to-anatase transformation. This is best evidenced by the fact that when the laser power was increased the Raman spectra change from one consistent with lepidocrocite to one consistent with anatase (inset FIG. 2).

The next task is to reconcile the XRD patterns (FIG. 1a) with the lepidocrocite structure. This is important here because, apart from the (200) peak at ≈48° 2□ and possibly the peak at ≈62° 2□□ all other peaks are not standard lepidocrocite issue.^{9, 14} The latter is typically characterized by a strong (103) peak at ≈26-28° 2□□ with a smaller (110) peak to its left.^{9, 14} More recently, Ma et al.¹⁵ published an XRD pattern they ascribed to lepidocrocite, with 4 peaks they indexed as (101), (004), (200) and one, at ≈62° 2□□ they did not index.

To model our structure, we made use of DFT calculations on lepidocrocite.¹⁶ The latter is comprised of 2 Ti-atom thick ribbons stacked along the b-direction (FIG. 1b). Half the O atoms are 4-fold coordinated; the other half 2-fold. The (200) peak in the XRD patterns is due to vertical plane labelled as such in FIG. 1b. As discussed below, the planes responsible for the peak at 62° 2□ are shown in FIG. 1c and indexed as (002). In our coordinate system (FIG. 1b), the peak at ≈26° 2□ is ascribed to the (110) planes (FIG. 1b). Most of the other peaks are 00/peaks characteristic of 2D materials. Note that in the ethanol washed samples (blue pattern in FIG. 1a) the order along the stacking direction is higher than in their LiCl-washed counterparts.

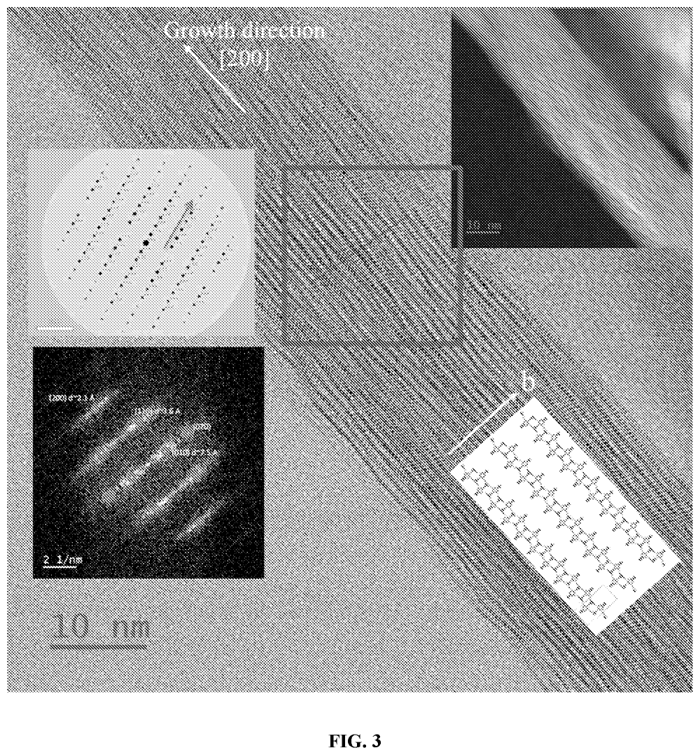

FIG. 3 presents an annular bright field (ABF) TEM image of a TiB2-derived bundle of NFs, together with a FFT of the center of the micrograph outlined by the blue square. To simulate the FFT, we started with the DFT-generated lepidocrocite structure¹⁶ and tilted it so the c-axis was the zone axis (FIG. 1b). The lepidocrocite layers were stacked along the b-axis (FIG. 1b) such that the growth direction was and, importantly, coincided with of the bundle axis (lower right inset FIG. 3). The stacking distance between the 2D layers was adjusted to match the (010) and

(020) spots on the FFT. The interlayer distance, henceforth referred to as d010, chosen was 7.5 Å. The other spots (top left inset in FIG. 3) were generated by the single-crystal diffraction module of the Crystal Maker software. Said otherwise only one adjustable parameter was used.

The agreement between the FFT spots and our simulated SAD—red circles in lower left inset—is excellent and suggests that the 110 and 200 d-spacings are 3.6 Å and 2.1 Å, respectively. The corresponding distances, derived from the XRD patterns for the (110) and (200) planes—henceforth referred as d110 and d200, respectively—were 3.5±0.8 Å and 1.89±0.01 Å.¹² Such a discrepancy in the d-spacings is not unexpected, especially when a FFT of an atomic-resolution STEM image is used. The position of the “diffraction spots” in the FFT is based on calibration of the underlying STEM image, which is affected by the accuracy of the underlying image calibration, scan distortions and image pixel size. The fact that in the DFT we model 2D lepidocrocite while experimentally we are dealing with 1DL could play a role. Also the fact that our material contains C,¹² but the DFT model does not, could prove important as we better understand where the C atoms reside. Needless to add, the XRD results are more accurate, but the symmetry of the diffraction peaks is consistent. Based on the d200 value, the a lattice parameter is 3.78 Å which is in slightly smaller than the 3.803 Å reported by Tominaka et al.¹⁷ who also used TMAH to make 2D lepidocrocite.

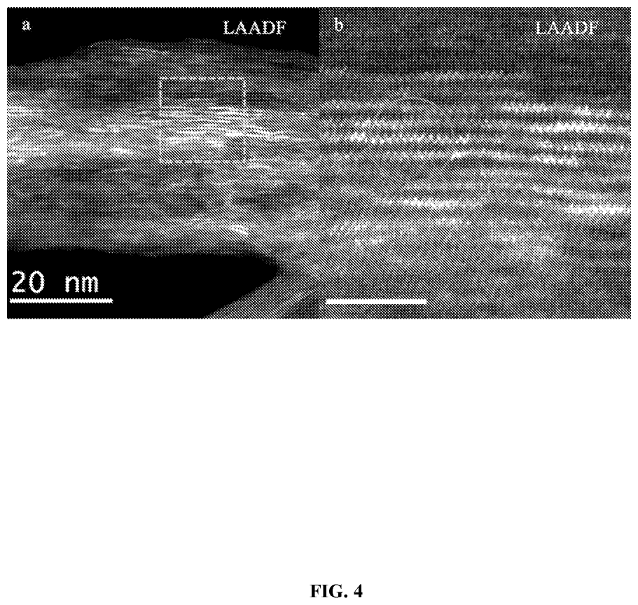

In the bright regions, where Ti-atomic columns presumably stack, it is possible to discern—as shown in FIG. 4—a zig-zag pattern to the Ti-atoms that is consistent with schematic shown in FIG. 1b.

Using the Scherer formula, we estimate the domain sizes along and to be 4.2, 7.3 and 3.4 nm, respectively. These dimensions are small compared to the micrograph shown in FIGS. 3, 4 and 5, and suggest that the order is on a finer scale than the relatively larger macroscopic features—viz. 2D flakes, fiber bundles etc.—observed.

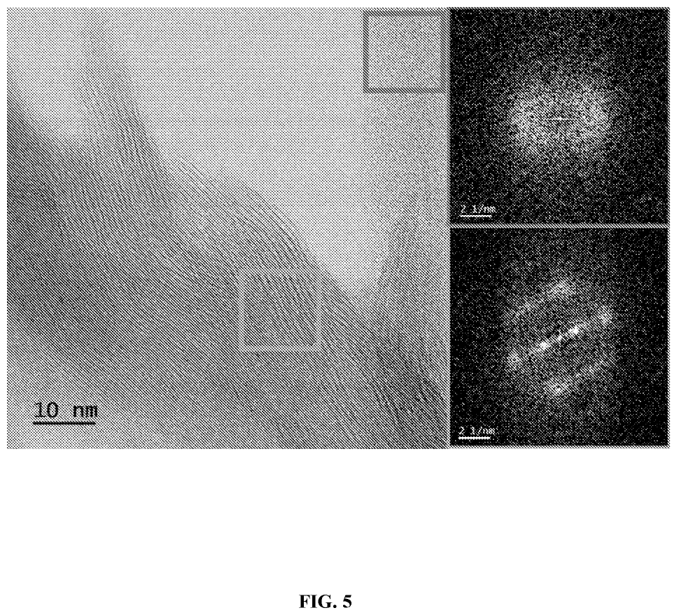

And while the “crystalline” regions were key for us to decipher the structure, it is also true that a good fraction of the bundles, or 2D flakes, are poorly crystallized. FIG. 5 two delineates regions enclosed by a blue and a green square. FFT pattern of the blue region—top left inset in FIG. 5—is clearly amorphous. The corresponding FFT (lower inset) of the green region resulted in a pattern that is the same as that shown in FIG. 3, but significantly less sharp.

At this juncture it is important to critically assess our proposed structure. Based on DFT calculations, the thickness of the 2-Ti atom ribbons, from outermost O to outermost O, is ≈4.1 Å (FIG. 1b). If the total interlayer distance is 7.5 Å, then the intergallery space is ≈3.4 Å. Why the (0h0) peaks, that are clearly seen in the FFT (FIG. 3), are missing from the XRD pattern is a mystery at this time. The origin of all other non-basal peaks can be traced to planes where the Ti atoms in one ribbon, or unit cell, are connected to ever increasing number of Ti atoms—numbered in FIG. 1b—in adjacent ribbons as shown in FIG. 1b. In our coordinate system, the first of these inclined planes is (110) with a d-spacing that is consistent with a XRD peak at 26° 2□.

The d-spacing of the ˜ 62° 2 peak does not match any of the (1n0) planes (FIG. 1b) and does not appear in simulated FFT shown in top left inset in FIG. 3. It thus must be associated with the c-axis. The DFT c-lattice parameter, LP, is ≈3.01 Å and its (002) d-spacing, d002, would be 1.5 Å, with a 2□ of ≈62.2°. Experimentally in XRD patterns, this peak appears at 61.0±0.4°,¹² corresponding to a c-LP of 3.04±0.06 Å. We thus ascribe this crystallographic peak to (002) reflections. Note there are two (002) reflections. The first is associated with the stacking of the NFs along the c-axis at 2□<20° (FIG. 1a). The second is crystallographic, and stems from the X-rays reflecting off the top of the ribbons shown in FIG. 1c, and appears at ≈62° 2□□

Based on the aforementioned results, we identified 2 of the 3 planes of our 1DL NFs; (100) and (001). What about the third surface, viz. (010)? In that surface, the Ti- and O-atoms are co-planar (FIG. 1c). If that surface is cut such that only 2 Ti layers remain, they would also project a zig-zag pattern (FIG. 1c), that would be quite difficult to differentiate from the (001) surface that also results in a zig-zag pattern (FIG. 4). This comment notwithstanding, from the TEM image shown in FIG. 3, and others, one can tentatively conclude that the thickness of (001) nanoribbons is of the order of ≈6 Å; their DFT width is 5.7 Å (FIG. 1c). Had this dimension been much wider, it is unlikely that the relatively homogenous microstructure shown in FIG. 3 would have been possible. As importantly if relatively large segments existed, they would have been crystalline and thus easily discernable in the TEM.

Along the same lines it is well-established in the MXene,¹⁸ and other 2D materials literature, that it is non-trivial to find multilayers, MLs, that are oriented “edge-on” since most of the 2D flakes lie with their basal parallel to the surface.¹⁹ Typically, it is mostly at MLs edges that flakes turn upwards exposing their basal planes in an edge-on configuration.¹⁸ Herein, the opposite is true; most regions are either poorly crystallized, amorphous or exhibit an “edge-on” formation (FIGS. 3, 4 and 5). When 2D lepidocrocite, with strong (101) peaks in XRD, is imaged in a TEM relatively large islands and lattice fringes are not difficult to find.^{15, 17} Their absence here strongly suggests they do not exist and what we have instead are 1DL NFs that self-assemble into “2D” flakes. This is important because if the NFs seen herein are truly 1D, then we are dealing with NFs that are ≈5×6 Å² in cross-section. It is important in this context to emphasize that we are not implying that 2D layers do not exist; the XRD patterns are clear. What we are saying is that the “2D” flakes are comprised of 1DL NFs that self-assembled into layers.

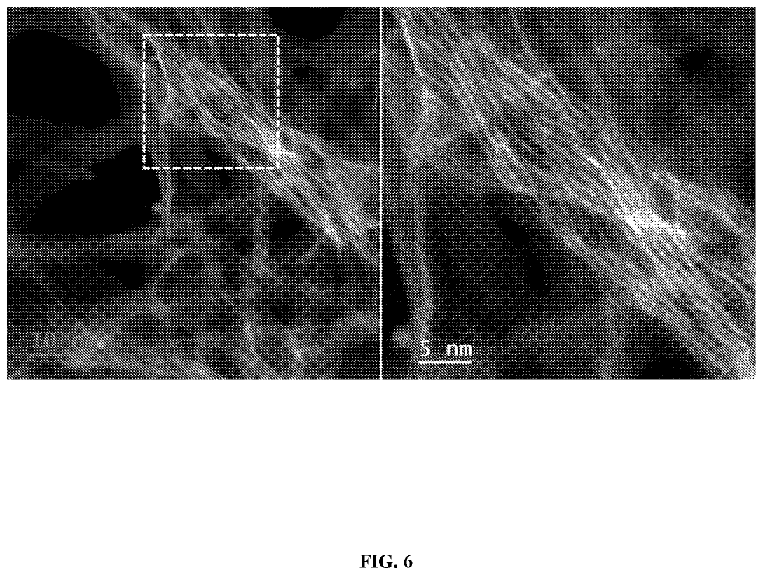

FIG. 6 further bolsters the conclusion that we are dealing with NFs. In this TiC-derived sample individual NFs can be readily discerned. These comments notwithstanding, we hereby acknowledge that what we see in our micrographs could, while unlikely, be edges of larger sheets that extend into the plane of the page.

It is important at this juncture to validate the conclusions reached above. Of the three distances, d₂₀₀, d₀₀₂ and d₁₀₁, only the first two are crystallographic. It is for this reason that for all materials produced to date—over 200 separate runs—the locations of the 200 peaks, at ≈48° 2□ in the XRD patterns was unchanged (FIG. 1a).¹² The same is true of the ≈62° 2□ peaks.¹² The locations of the (110) peaks, on the other hand, are a function of the surrounding media (FIG. 1a) and thus cannot be crystallographic. Another important observation consistent with this notion is that the distance between NFs along the red line plotted in FIG. 3 is ≈7 Å, which is comparable to the 7.5 Å used to adjust theory to the FFT pattern.

Compared to 2D materials with one stacking direction, here there are two; one along the (010) direction or b-axis (lower right inset in FIG. 3); the other is out-of-the plane of the page (along the c-axis) that is responsible for the low angle reflections labelled (00l) in FIG. 1. Not much information can be gleaned from the STEM images about the c-axis spacing or stacking. Not surprisingly, that spacing is also a function of the nature of the cations surrounding the NFs as

shown by peaks labelled (00l) in FIG. 1. Note, most of the peaks, and the strongest ones, are (00l) peaks. This is especially manifest when the y-axis is plotted linearly and not logarithmically.

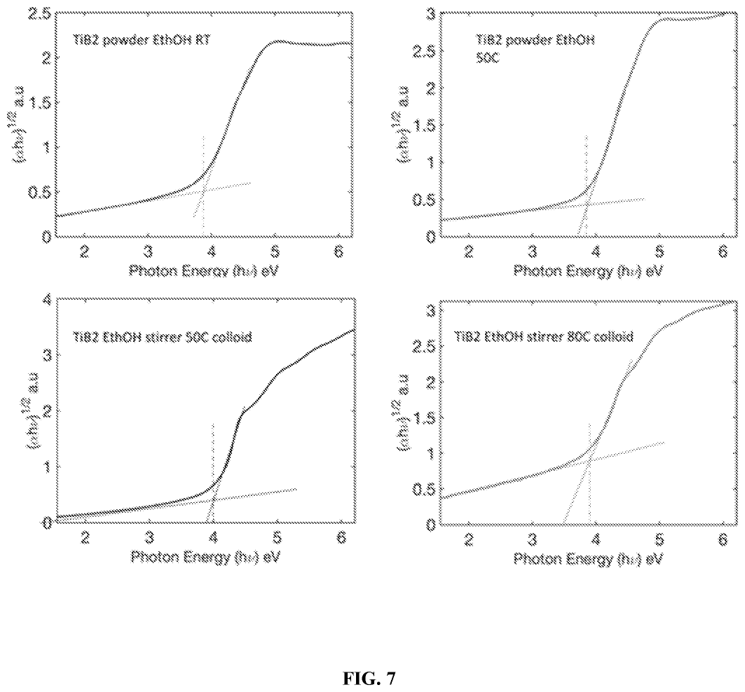

Lastly, and while the washing protocol changes the spacing between NFs, these variations do not affect the band gap. Tauc plots (FIG. 7) confirm the presence of as indirect band gap at ≈4 eV reported on earlier.¹² We note in passing and as discussed in our previous work,¹² this band gap energy is a record for a TiO₂-based material made via a bottom up approach and is an independent confirmation of a quantum confinement effect. There are numerous reports in the literature on 1D TiO₂-based materials. As far as we are aware, however, none of them report a quantum size effect on the band gap.

In conclusion, the 1D NFs produced by reacting TiB₂ and TiC powders in TMAH at 80° C. for 5 days crystalize in the lepidocrocite TiO₂ structure. The NFs grow in the

direction and stack along the b-direction in the plane that the NFs self-assemble to either create bundles (FIG. 3) or larger 2D flakes shown in our previous work.¹² And while the “crystalline” regions are key for us to understand the structure, it is also true that a good fraction of the bundles, or 2D flakes, are poorly crystallized. Some areas appear amorphous.

Regardless of how well the NFs are self-assembled or not, if we assume them to be 6×5 Å² in cross-section, their theoretical specific surface area would be >1700 m²/g. This is an extraordinary number for a Ti-containing material and partially explains some of the remarkable properties these materials exhibit. The fact that the process to make them is inexpensive, highly scalable—we routinely make 100 g batches in a laboratory setting—and the precursors powders, such as TiC, TiB₂, Ti-containing MAX phases, are earth abundant, and non-toxic, bodes well for their large-scale application in myriad fields.

### Methods

### Materials Synthesis and Processing

Samples of 1DL NFs prepared by shaking TiB2 (Thermo Scientific, -325)

powders with tetramethyl ammonium hydroxide aqueous solution, TMAH (Alfa Aesar, 25 wt. % in DI water, 99.9999%) at 80° C. for 5 days using a temperature-controlled incubator/shaker. In all cases, the Ti: TMAH mole ratio was kept at 0.6. After reaction, the resulting powders were washed with ethanol (Decon Lab Inc., 200 proof) till pH was ≈7. The powders were then dehydrated in open air at 50° C. overnight. To explore any potential effect of the drying temperature, another sample, from the same batch, was dehydrated at room temperature, RT instead.

To assess capability of ion exchange, ethanol-washed sediments were further stirred, while wet, 3 subsequent times each of 6 h in one of the following salt solutions: LiCl 0.5M, LiCl 5M, NaCl 0.5M, or NaCl 5M and then rinsed with DI water 3 times to remove any unreacted salt or reaction products. All salts purchased from Alfa Aesar with >99% purity. The LiCl- and NaCl-washed powders were then air-dried at 50° C., similar to above.

To compare shaker-processed samples to those produced using magnetic stirring,²⁰ in one case TiB₂ powders were magnetically stirred, at 300 rpm, in a TMAH solution following the abovementioned conditions of mole ratio, temperature, and time. After reaction, the resulting slurry washed 6 times with ethanol till pH was ≈7, redispersed in DI water, shook for 5 min, then centrifuged at 3500 rpm for 30 min. The resulting colloidal suspension was then filtered using vacuum-assisted filtration to produce a filtered film that was dried at 50° C. in open air overnight.

The Raman spectra shown in FIG. 2 were obtained for the following six samples: ethanol washed, LiCl 0.5M, LiCl 5M, NaCl 0.5M, NaCl 5M and the magnetically stirred sample.

### X-ray Diffraction

XRD patterns were acquired using a diffractometer (Rigaku MiniFlex) operated with Cu Kα radiation (40 kV and 15 mA) in the 2-65° 2θ range with step size of 0.02° and a dwell time of 1 s. All XRD patterns obtained from powders dried overnight at 50° C. in open air.

### Raman Spectroscopy

Two sets of Raman spectra were obtained in two different labs. At Drexel

University, Raman spectra were collected at room temperature in air. Measurements were done with an inverted reflection mode Renishaw In Via (Gloucestershire, U.K.) instrument equipped with 63×(NA=0.7) objectives and a diffraction-based room-temperature CCD spectrometer. Ar+ laser (514 nm) was used and the laser power was kept in the ˜0.5-1.5 mW range.

In another set, obtained at Fayetteville University, suspensions of TiB₂-derived material QDN with a concentration of 10 mg/mL were prepared with solvents of deionized (DI) water (Millipore), isopropyl alcohol (>99.7%, Sigma Aldrich) and dimethyl sulfoxide (DMSO, 99.9%, Sigma Aldrich) and were drop-cast onto a microscope slide and allowed to air dry at room temperature, RT, for 24 hours. Raman spectra were collected at room temperature using an XploRA PLUS confocal Raman microscope (Horiba Scientific, Piscataway, NJ, USA) with a 250 mm focal length spectrometer in a backscatter geometric configuration. The spectrometer was first calibrated using a silicon chip with excitation by an air-cooled 532 nm solid state laser (100 mW) and using a 100× (NA=0.9 and WD =0.21 mm) objective to obtain a 1 μm spot size. A 1200 gr/mm grating was used and the scattered light was collected with a thermoelectrically (TE) air-cooled charge-coupled device (CCD) detector with 1024×256 pixels for a spectral resolution of 1 cm⁻¹. A neutral density (ND) filter wheel was used to attenuate the laser power to 10%, 25%, 50%, or 100%, and spectra were acquired at a lower power (10%) or higher. Raman spectra were collected in the 75-1200 cm⁻¹ range with 2 s integration time and 64 accumulations. The LabSpec 6 software was used to fit the collected Raman spectrum according to the Gaussian-Lorentzian function to obtain the peak positions and their intensities.

### Transmission Electron Microscopy

Atomic scale characterization was conducted using an aberration-corrected, cold-field emission TEM (JEOL ARM200CF) operated at 200kV primary electron energy.²¹ Imaging was conducted with the emission current at 15 μA and an electron probe semi-convergence angle of 24 mrad resulting in an electron probe size of approximately 80 pm. Annular bright field (ABF) imaging,²²⁻²³ which is a coherent imaging technique, was conducted using an outer angle of 23 mrad and an inner angle of 11 mrad. For low angle annular dark field (LAADF) imaging the inner and outer angles were 30 mrad and 120 mrad, respectively. HAADF images²⁴ were collected with 68 mrad and 280 mrad inner and outer detector angles, respectively. The primary contrast mechanism for HAADF imaging is related to the square of the average atomic number and the total thickness of the atomic columns.²⁵

The TEM samples were prepared by dispersing nanofilament powders in 5 ml of methanol. The solution was drop casted onto a 3 mm copper mesh coated with a lacey carbon film and allowed to dry for an hour. The TEM grid was then loaded onto a plasma cleaned double tilt holder and inserted into the microscope column.

### REFERENCES

Kumar, S. G.; Devi, L. G., Review on Modified TiO2 Photocatalysis under UV/Visible Light: Selected Results and Related Mechanisms on Interfacial Charge Carrier Transfer Dynamics. The Journal of Physical Chemistry A 2011, 115 (46), 13211-13241.

Schaub, R.; Thostrup, P.; Lopez, N.; Lægsgaard, E.; Stensgaard, I.; Nørskov, J. K.; Besenbacher, F., Oxygen vacancies as active sites for water dissociation on rutile TiO2 (110). Physical Review Letters 2001, 87 (26), 266104.

Wang, A. S. D.; Sasaki, T., Titanium Oxide Nanosheets: Graphene Analogues with Versatile Functionalities. Chem. Rev. 2014, 114, 9455-9486.

Bourikas, K.; Kordulis, C.; Lycourghiotis, A., Titanium Dioxide (Anatase and Rutile): Surface Chemistry, Liquid-Solid Interface Chemistry, and Scientific Synthesis of Supported Catalysts. Chemical Reviews 2014, 114 (19), 9754-9823.

Wang, X.; Li, Z.; Shi, J.; Yu, Y., One-dimensional titanium dioxide nanomaterials: nanowires, nanorods, and nanobelts. Chemical reviews 2014, 114 (19), 9346-9384.

Ramos-Delgado, N. A.; Gracia-Pinilla, M. A.; Mangalaraja, R. V.; O'Shea, K.; Dionysiou, D. D., Industrial synthesis and characterization of nanophotocatalysts materials: titania. Nanotechnology Reviews 2016, 5 (5), 467-479.

Fang, W.; Xing, M.; Zhang, J., Modifications on Reduced Titanium Dioxide Photocatalysts: A review. J. Photochem. Photobiolog C: Photochem. Rev. 32 2017, 21-39.

Saeed, M.; Muneer, M.; Akram, N., Photocatalysis: an effective tool for photodegradation of dyes—a review. Environmental Science and Pollution Research 2021, 1-19.

Sasaki, T.; Watanabe, M.; Michiue, Y.; Komatsu, Y.; Izumi, F.; Takenouchi, S., Preparation and Acid—Base Properties of a Protonated Titanate with the Lepidocrocite-like Layer. Chem. Mater. 1995, 7, 1001.

Sasaki, T.; Watanabe, M., Semiconductor nanosheet crystallites of quasi-TiO2 and their optical properties. J. Phys. Chem. B. 1997, 101 (49), 10159-10161.

Zhou, W.; Umezawa, N.; Ma, R.; Sakai, N.; Ebina, Y.; Sano, K.; Liu, M.; Ishida, Y.; Aida, T.; Sasaki, T., Spontaneous Direct Band Gap, High Hole Mobility, and Huge Exciton Energy in Atomic-Thin TiO2 Nanosheet. Chemistry of Materials 2018, 30 (18), 6449-6457.

Badr, H.; El-Melegy, T.; Carey, M.; Natu, V.; Hassig, M. Q.; Johnson, C.; Qian, Q.; Li, C. Y.; Kushnir, K.; Ulloa, E. C.; Titova, L. V.; Martin, J.; Grimm, R. L.; Pai, R.; Kalra, V.; Karmakar, A.; Liang, K.; Naguib, M.; Wilson, O.; Magenau, A. J. D.; Montazeri, K.; Zhu, Y.; Cheng, H.; Torita, T.; Koyanagi, M.; Yanagimachi, A.; Ouisse, T.; Barbier, M.; Wihlem, F.; Rogalev, A.; Persson, P.; Rosen, J.; Hu, Y.-J.; Barsoum, M. W., Bottom-up, scalable synthesis of anatase nanofilament-based two-dimensional titanium carbo-oxide flakes. Mater. Today 2022, 54, 8-17.

Hu, W.; Li, L.; Li, G.; Liu, Y.; Withers, R. L., Atomic-scale control of TiO6 octahedra through solution chemistry towards giant dielectric response. Scientific reports 2014, 4 (1), 1-9.

Esmat, M.; Farghali, A. A.; El-Dek, S. I.; Khedr, M. H.; Yamauchi, Y.; Bando, Y.; Fukata, N.; Ide, Y., Conversion of a 2D Lepidocrocite-Type Layered Titanate into Its 1D Nanowire Form with Enhancement of Cation Exchange and Photocatalytic Performance. Inorganic Chemistry 2019, 58 (12), 7989-7996.

Ma, J.; Reeves, K. G.; Porras Gutierrez, A.-G.; Body, M.; Legein, C.; Kakinuma, K.; Borkiewicz, O. J.; Chapman, K. W.; Groult, H.; Salanne, M.; Dambournet, D., Layered Lepidocrocite Type Structure Isolated by Revisiting the Sol-Gel Chemistry of Anatase TiO2: A New Anode Material for Batteries. Chemistry of Materials 2017, 29 (19), 8313-8324.

Reeves, K. G.; Ma, J.; Fukunishi, M.; Salanne, M.; Komaba, S.; Dambournet, D., Insights into Li+, Na+, and K+ Intercalation in Lepidocrocite-Type Layered TiO2 Structures. ACS Applied Energy Materials 2018, 1 (5), 2078-2086.

Tominaka, S.; Yamada, H.; Hiroi, S.; Kawaguchi, S. I.; Ohara, K., Lepidocrocite-type titanate formation from isostructural prestructures under hydrothermal reactions: observation by synchrotron X-ray total scattering analyses. ACS omega 2018, 3 (8), 8874-8881.

Naguib, M.; Kurtoglu, M.; Presser, V.; Lu, J.; Niu, J.; Heon, M.; Hultman, L.; Gogotsi, Y.; Barsoum, M. W., Two Dimensional Nanocrystals Produced by Exfoliation of Ti3AlC2. Advan. Mater. 2011, 23, 4248-4253.

Coleman, J. N.; Lotya, M.; Arlene O′Neill; Bergin, S. D.; King, P. J.; Khan, U.; Young, K.; Gaucher, A.; De, S.; Smith, R. J.; Shvets, I. V.; Arora, S. K.; Stanton, G.; Kim, H.-Y.; Lee, K.; Kim, G. T.; Duesberg, G. S.; Hallam, T.; Boland, J. J.; Wang, J. J.; Donegan, J. F.; Grunlan, J. C.; Moriarty, G.; Shmeliov, A.; Nicholls, R. J.; Perkins, J. M.; Grieveson, E. M.; Theuwissen, K.; McComb, D. W.; Nellist, P. D.; Nicolosi, V., Two-Dimensional Nanosheets Produced by Liquid Exfoliation of Layered Materials. Science 2011, 331, 568

Badr, H. O.; El-Melegy, T.; Carey, M.; Natu, V.; Hassig, M. Q.; Johnson, C.; Qian, Q.; Li, C. Y.; Kushnir, K.; Colin-Ulloa, E.; Titova, L. V.; Martin, J. L.; Grimm, R. L.; Pai, R.; Kalra, V.; Karmakar, A.; Ruffino, A.; Masiuk, S.; Liang, K.; Naguib, M.; Wilson, O.; Magenau, A.; Montazeri, K.; Zhu, Y.; Cheng, H.; Torita, T.; Koyanagi, M.; Yanagimachi, A.; Ouisse, T.; Barbier, M.; Wilhelm, F.; Rogalev, A.; Björk, J.; Persson, P. O. Å.; Rosen, J.; Hu, Y.-J.; Barsoum, M. W., Bottom-up, scalable synthesis of anatase nanofilament-based two-dimensional titanium carbo-oxide flakes. Mat. Today 2022, 54, 8-17.

Klie, R. F.; Gulec, A.; Guo, Z.; Paulauskas, T.; Qiao, Q.; Tao, R.; Wang, C.; Low, K. B.; Nicholls, A. W.; Phillips, P. J., The new JEOL JEM-ARM200CF at the University of Illinois at Chicago. Crystal Research and Technology 2014, 49 (9), 653-662.

Phillips, P. J.; Klie, R. F., On the visibility of very thin specimens in annular bright field scanning transmission electron microscopy. Applied Physics Letters 2013, 103 (3).

Findlay, S. D.; Shibata, N.; Sawada, H.; Okunishi, E.; Kondo, Y.; Yamamoto, T.; Ikuhara, Y., Robust atomic resolution imaging of light elements using scanning transmission electron microscopy. Applied Physics Letters 2009, 95 (19), 191913-3.

Pennycook, S. J.; Boatner, L. A., Chemically Sensitive Structure-Imaging With A Scanning-Transmission Electron-Microscope. Nature 1988, 336 (6199), 565-567.

Nellist, P. D.; Pennycook, S. J., Incoherent imaging using dynamically scattered coherent electrons. Ultramicroscopy 1999, 78 (1-4), 111-124.

### Additional Disclosure—II

Titania (TiO₂) nanostructures have been, and remain, of significant research interest due to their unique physical and chemical properties as well as their potential application in a wide range of fields including paint pigment, catalysis, photocatalysis, photoluminescence, gas sensors, solar and fuel cells among many others. Amongst commercially available nanostructured titania, Evonik's Aeroxide TiO₂ P25 (formerly sold by Degussa), and hereafter referred to as P25, is used. P25 synthesized via flame pyrolysis of TiCl₄, is attractive because of its high photocatalytic activity.

In many ways, P25 has been, and is still considered the gold standard for TiO₂-based catalytic and photocatalytic applications. Its major drawback, however, is its cost; flame pyrolysis is a relatively expensive process. Arguably, had P25 been cheaper it would have found many more applications. Thus, we developed a significantly cheaper process to make one dimensional (1D) titania, that we have shown performs better than P25 in a number of applications.

The recipe entails reacting precursor powders with tetramethylammonium hydroxide (TMAH) aqueous solutions—in polyethylene bottles—in the 50° C. to 85° C. temperature range for a few days. In one case, we reacted 5 different Mn-containing powders, such as Mn₃O₄, Mn₂O₃, MnB, etc., in TMAH aqueous solutions for a few days and converted them into birnessite-based two-dimensional (2D) sheets with thicknesses of 2±0.4 nm that were ≈200 nm across. These 2D birnessite sheets were remarkably crystalline. They, in turn, demonstrated enhanced electrochemical reactivity for both reversible O₂ electrocatalysis and supercapacitor applications.

Following the same protocol, immersing FeB powders in alkaline aqueous solutions (TMAH; tetramethylammonium hydroxide, TBAH; or potassium hydroxide, KOH) produced ferromagnetic Fe₃O₄ nanoparticles with an average particle size of ˜15 nm.

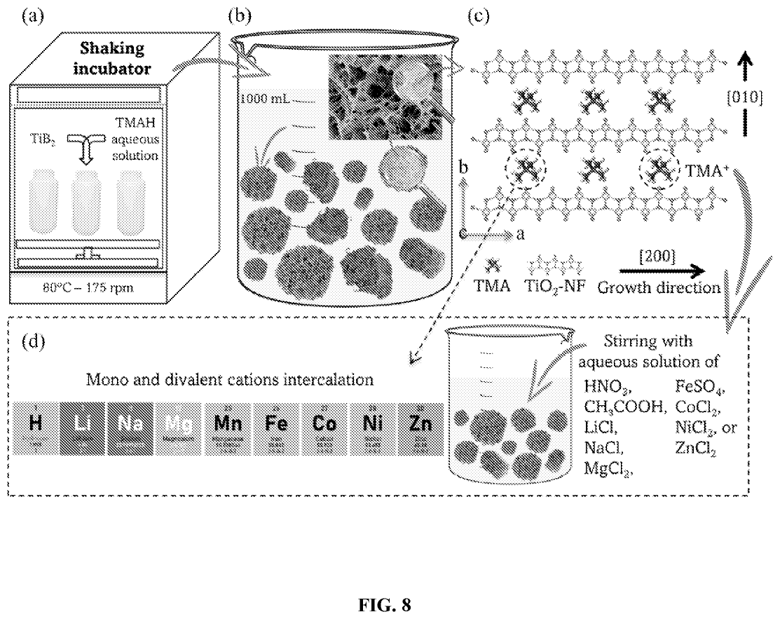

In another example, we converted, cheap, earth abundant, water-insoluble Ti-bearing precursors including TiC, TiN, TiB₂, among others, into 1D nanofilaments, NFs. The recipe entails reacting Ti-bearing precursor powders with, again, TMAH aqueous solutions—in polyethylene bottles—in the 50° C. to 85° C. temperature range for a few days. We concluded that the 1D NFs crystallize in a lepidocrocite-type TiO₂-based structure (FIG. 8c). Henceforth, these lepidocrocite 1D NFs will be referred to 1DL. The cross-sections of our 1DL NFs are ≈5×7 Å². It is the extreme size led us to conclude that a quantum size effect was responsible for the record band gap energy, E_{g}≈4eV, for a bottom-up processed, titania-based material.

We concluded several studies of our 1DLs showing them to be unique and better performing than P25. We showed that the photochemical hydrogen production rates when exposed to the equivalence of one sun, were about an order of magnitude higher than P25 tested under identical conditions. In the field of water purification, we showed that our 1DLs can adsorb record values of uranium (U⁴⁺) rendering water contaminated by this actinide potable. Lastly, composites of our 1DLs with a repairable, dynamic covalent thiolyne network resulted in a 500 times increase in the modulus at 60 wt % filler when compared to pristine polymer.

After reaction and washing with ethanol, EtOH, to ≈pH 7 and then washing with water, pseudo-two-dimensional, p-2D were produced upon filtration. The reason we refer to them as ‘pseudo’ is because the flakes—comprised of 1DL NFs—are only apparently 2D. We showed that these p-2D flakes are present in the colloid, even at short reaction times. It follows that water exerts a strong driving force aligning the 1DLs

normal to their growth direction. This self-alignment first leads to the formation of nano-bundles and μ-fibers that in turn self-align into p-2D flakes. Regardless of the experimental conditions or final morphologies, the 1DL NFs remain the essential building blocks.

Here, we show that if the 1DL NFs are dried while in ethanol (i.e. without dispersing in water) they form spherical, mesoporous particles, henceforth referred to as MPPs, with diameters that are comparable to those of the precursor powder. In this work we: i) Report on large scale synthesis (100 g batches) of MPPs that are comprised of 1DL NFs; ii) Shed light on the mechanisms that lead to MPPs formation; iii) Show that, like in other layered titanates, the space between the NFs is eminently ion exchangeable. To that effect, we can readily replace the TMA⁺ cations present after the reaction stage by H⁺, Li⁺, Na⁺, Mg²⁺, Mn²⁺, Fe²⁺, Ni²⁺, Co²⁺, or Zn²⁺ cations (see schematic in FIG. 8d). iv) Measure the surface charges, and hydrodynamic radii of MPPs intercalated with TMA⁺ or Li⁺ ions.

The starting precursor chosen is TiB₂ because, compared to TiC and TiN, it is the most reactive. A large batch of TiB₂ powders can be almost fully converted to 1DLs in ≈3 d at 80° C.

### Results and Discussion

Precursors were reacted with TMAH using hot plates and magnetic stirrers. To prepare batches as large as 100 g at once we used a temperature-controlled shaking incubator. The experimental details can be found in the experimental procedures section. In brief, we shake 100 g of TiB2 commercial powders with ˜1 L of a 25 wt. % TMAH aqueous solution at 80° C. for 1 to 5 days, d, in a temperature-controlled shaking incubator (FIG. 8a). In one set of experiments, the resulting powders were washed with EtOH multiple times using an overhead mixer till the pH was ˜7 (FIG. 8b), before they are left to dry at 50° C. in open air.

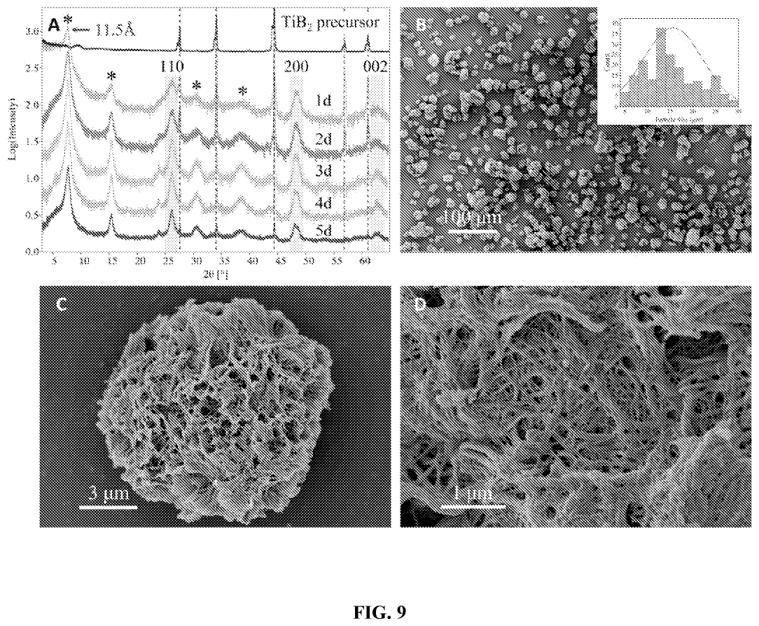

Preliminary results indicated that when the EtOH washed samples were placed in water the MPPs did not retain their morphology. If, however, the TMA⁺ cations were replaced with Li⁺ they did. To explore this idea, in one set of experiments, EtOH washed powders were stirred—while wet—in aqueous salt solutions of 0.5 M LiCl, 5 M LiCl, 0.5 M NaCl or 5 M NaCl (FIG. 9d). The powders were then rinsed with DI water a few times to remove any residual salts before drying at 50° C. in open air.

To assess the capability of intercalating various mono- and divalent cations between the NFs, both the EtOH and the EtOH/LiCl washed powders were further treated in one of the following aqueous solutions (FIG. 8d): i) 0.1 M nitric acid, HNO₃, 0.5 M acetic acid or, ii) 0.02 M aqueous solution of one of the following salts: MgCl₂, MnCl₂, FeSO₄, CoCl₂, NiCl₂ or ZnCl₂. In all cases, after being immersed in the salt solutions the powders were washed a few times with DI water and dried at 50° C. in open air for 24 h.

### Characterization of 1DL NFs

Before proceeding further, one can review the X-ray diffraction (XRD) signature of our 1DL NFs. The reaction time dependencies of the XRD patterns—on a log scale—are shown in FIG. 9a. In a typical 1DL XRD patterns (FIG. 9a), three types of peaks exist. The first are due to unreacted precursor—TiB₂ in this case—denoted by dashed black lines in FIG. 9a. These are useful in that they can be used as internal standards. The second, 010 peak at low 2q angles—and its higher, 0k0 reflections denoted by asterisks—reflect the d-spacing values between NFs stacked along the b-direction. Like in other 2D materials, these peak locations are strong functions of what cations are intercalated between them. Crucially, here the distance is not between flakes but between NFs. Based on the results shown in FIG. 9a, it clear that after the first day, the d-spacings are no longer functions of reaction time.

The (110) peak located, around ˜26° 2q (denoted by grey band in FIG. 9a) is a weak function of the cations between the NFs. The last, and most fundamental peaks, are those at 2q values of ˜48° and 62°—denoted by red bands in FIG. 9a-indexed as 200 and 002 of the lepidocrocite structure, respectively. These peaks are useful because they are crystallographic in nature and should—as confirmed herein—be totally independent of what cations are in the system. It is from these 2q values that we obtain the a- and c-lattice parameters of lepidocrocite, viz. 3.7 Å and 2.9 Å.

As just noted, FIG. 9a shows the time dependencies of the XRD patterns of the TiB₂ precursor powder (top pattern in FIG. 9a) as well as those reacted at 80° C. for 1 d to 5 d, shown from top to bottom in FIG. 9a. As the reaction times increased from 1 to 3 d the intensity of the TiB₂ diffraction peaks gradually decreased, whereas the 1DL ones became dominant. The latter is again recognized by the two red bands in FIG. 9a, and the low angle 010 peak at 9° 2q (and its higher order peaks). From the latter, the distance between the 1DL NFs is calculated to be 11.5 Å. These results suggest that after 3 d, the conversion of the precursors into 1DL powders is complete. However, to minimize the fraction of unreacted precursor we ran the reaction for 5 d. All characterizations were carried out on powders reacted for 5 d (blue curve in FIG. 9a).

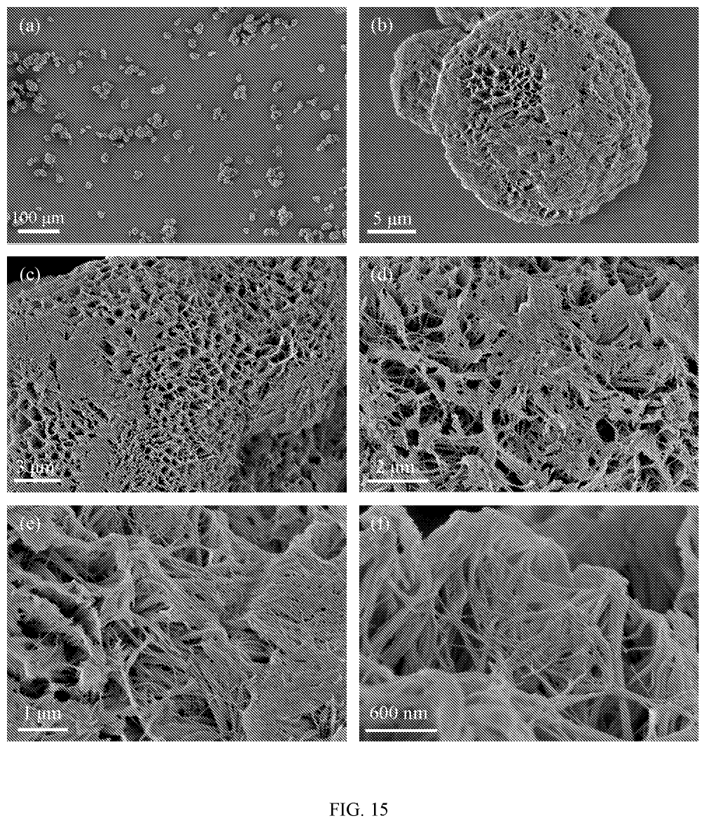

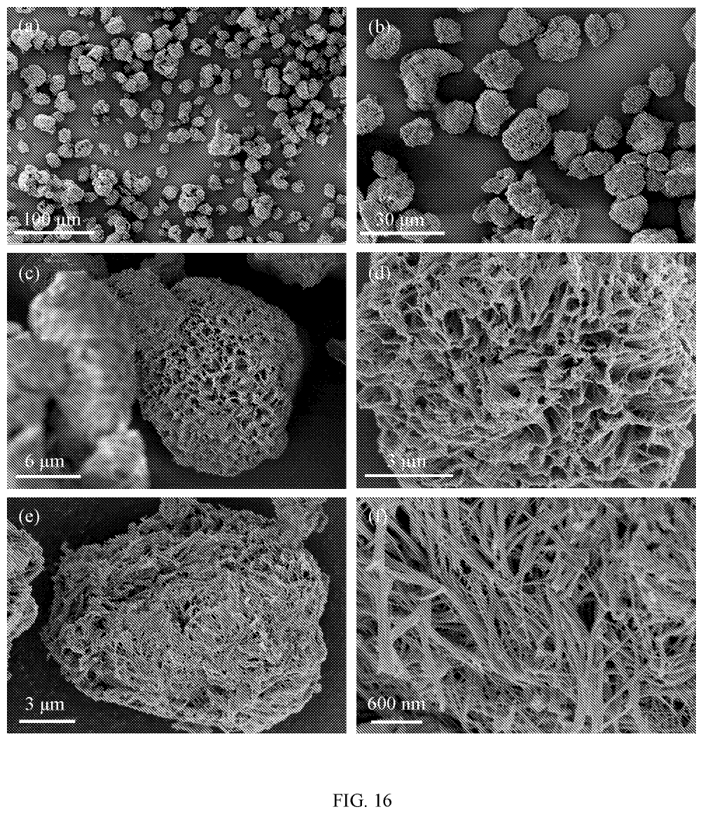

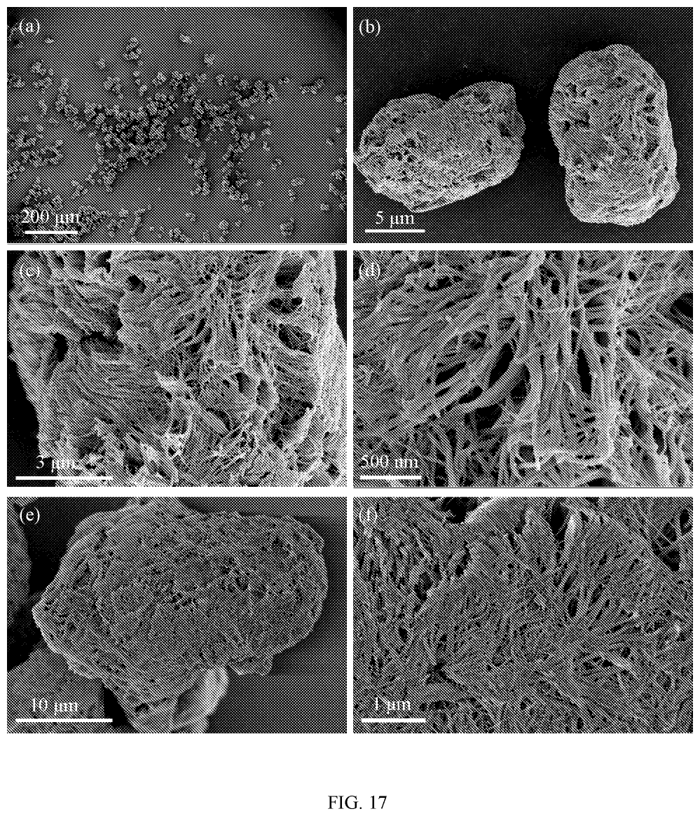

Scanning electron microscope (SEM) micrographs of typical MPPs after EtOH washing to pH 7, are shown in FIGS. 9b-d. At the millimeter scale, the powders appeared to be well dispersed with little to no aggregation observed (FIG. 9b). At higher magnification, the MPPs are porous, mostly spherical, with an average size of ˜13 μm (inset in FIG. 9b) and comprised of entangled 1DL NF bundles (FIGS. 9c and d). The shape and size of the MPPs are quite consistent over 50 different batches prepared and characterized to date. Other micrographs are shown in FIGS. 15-17. To summarize this section: After washing with EtOH, the 1DL NFs self-assemble into separate, non-agglomerated, free-flowing MPPs, in the 5 to 30 μm particle size range (FIG. 2b and inset).

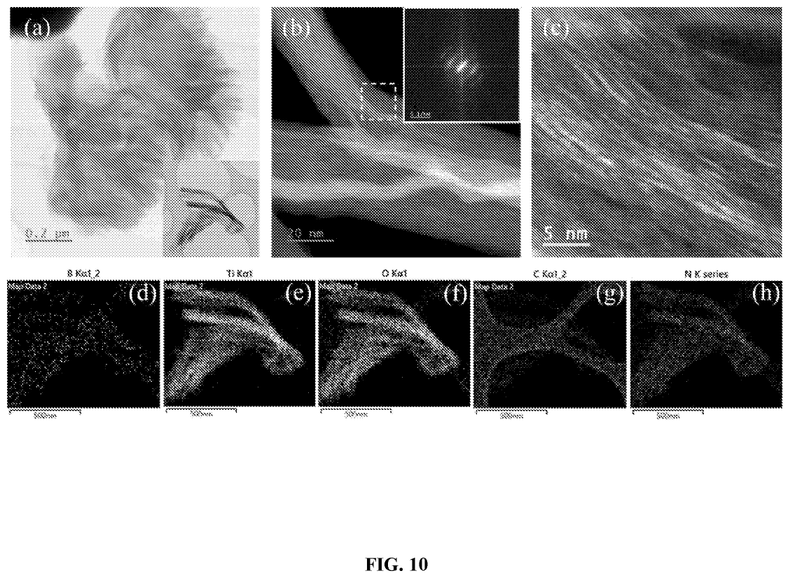

To better understand the MPP structure, we imaged them in a HR-STEM (FIGS. 10a-c). If one assumes that the fiber bundle shown in FIG. 10a is a single MPP, then its diameter is about 1 μm. At higher magnifications, it is obvious that the bundle is, in turn, comprised of a multitude of 1DL NFs (FIGS. 10b and c). A low angle annular dark field (LAADF) image (FIG. 10c) shows that the building unit remains NFs, 2 Ti-atoms wide, with a zigzag pattern. Fast Fourier transforms (FFT) pattern of the bundles (inset in FIG. 10b) resulted in 2 main arcs—confirming the 1D nature of our NFs—with d-spacings corresponding to XRD peaks at 2θ values of ˜26° and ˜48° (grey and red dashed bands in FIG. 9a). Also, the arcs bisect the growth direction.

Turning to the composition of the 1DL bundles, we obtained STEM-EDS maps of the MPP shown in inset in FIG. 10a. Only trace amounts of B (<1%) were detected (FIG. 10d) which is consistent with the almost complete conversion of TiB₂ into 1DL NFs and the effectiveness of our process in washing out any B-containing reaction products. From a scaling point of view, these powders were neither centrifuged nor filtered. Note the uniform distribution of Ti and O atoms (FIGS. 10e-f). The calculated atomic percent ratios of Ti and O are 24.5% and 49.5% respectively, consistent with a TiO₂ stoichiometry. The lacey carbon support is visible in the C map (FIG. 10g) and overshadows the C in the 1DL. The uniform distribution of N on the MPPs supports the fact that TMA⁺ ions are intercalated between the NFs (FIG. 10h). It is difficult to quantify the N amount present because of the large overlap between the N K- and the Ti L-edges.

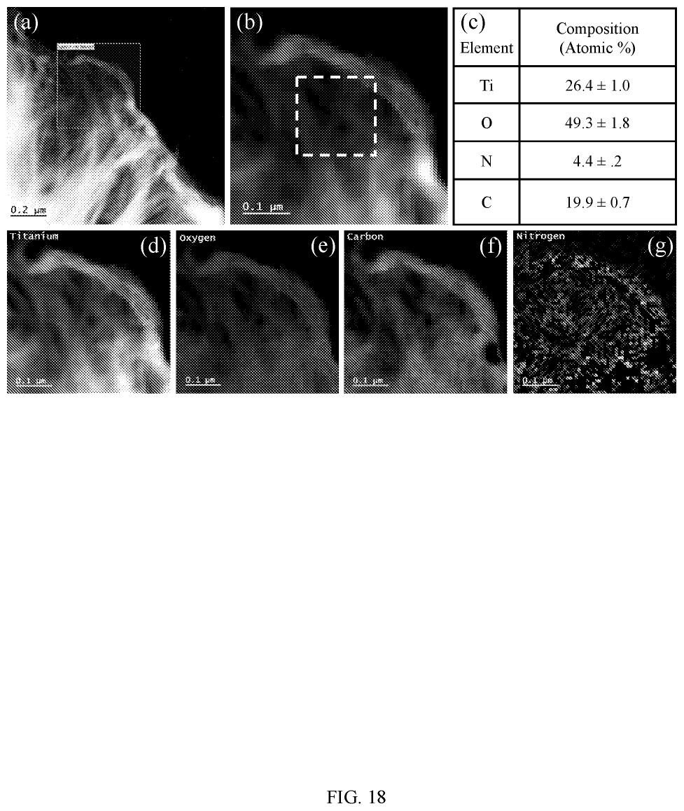

To solve this problem, and obtain a better handle on the C-content, we acquired electron energy loss spectroscopy (EELS) spectra where the Ti and N peaks are easily distinguishable, and the elemental compositions can be calculated using Hartree-Slater cross section models. To further reduce the contribution of surface hydrocarbons, we in situ cooled the sample using a liquid nitrogen, N₂, cold stage. The elemental maps derived from core loss spectra (FIG. 18) show Ti and O concentrations, again, consistent with a TiO₂ stoichiometry. The C to N ratio is 4.5, which is consistent with the expected ratio of 4 for TMA cations.

### Chemical Stability and Cationic Exchange of 1DL NFs

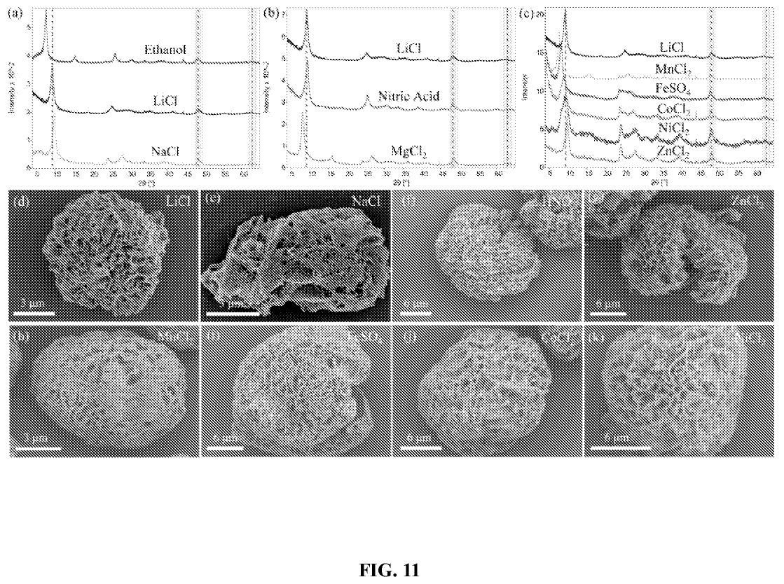

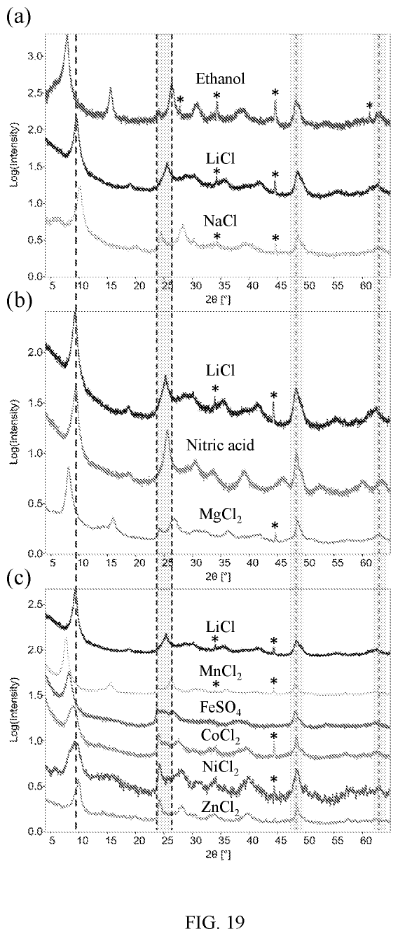

We established that we could readily ion exchange the TMA⁺ cations—present after EtOH washing to pH ≈7—with Li⁺. Here we show that the Li⁺ cations can, in turn, be replaced by the following cations: H⁺, Na⁺, Mg²⁺, Mn²⁺, Fe²⁺, Co²⁺, Ni²⁺, and Zn²⁺. The XRD patterns after ion exchange are plotted in FIGS. 11a-c. Typical SEM micrographs of the MPPs are shown in FIGS. 11d-k. FIGS. 19a-c plot the same data shown in FIG. 11a-c, respectively, but on a log scale.

As noted in the experimental section, after ion exchange all powders were rinsed with DI water multiple times and allowed to dry at 50° C. in open air before any further characterization. In all cases, the absence of all but 1DL XRD peaks (FIGS. 11a-c and 19a-c) confirm that we successfully eliminated most of the unreacted salts and unwanted reaction products. This also implies that the unwanted reaction products are water soluble.

After EtOH washing (blue patterns in FIGS. 11c and 19a), the first peak at ˜7.5° 2θ, corresponds to a 010 basal reflection for which the d ˜11.5 Å. This d-spacing is a measure of the thicknesses of the NFs along the b-direction together with any intercalated ions and/or water. Because we know from DFT calculations that the thickness of one NF along the b-direction is ≈7 Å, it follows that the TMA⁺ and H₂O thickness is ≈4.5 Å, which is reasonable.

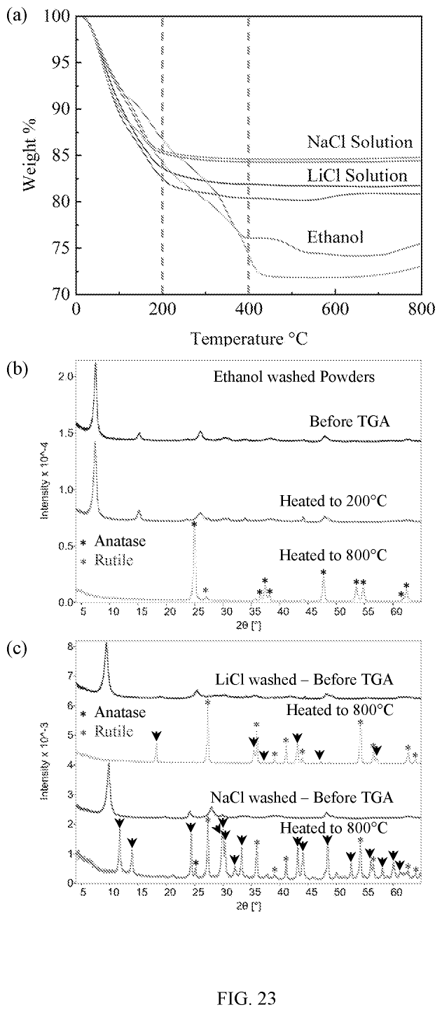

After washing with LiCl or NaCl solutions (black and green patterns, respectively, in FIGS. 11a and 19a), the d-spacing shrinks to 9.5 Å and 9.0 Å, confirming that the TMA⁺ ions were successfully exchanged with Li⁺or Na⁺ ions, respectively. The slightly higher d-spacing associated with Li⁺-intercalated powders, as compared to Na⁺, probably reflects the former's slightly larger hydration shell (see TGA results in FIG. 23). Again, assuming the thickness of one NF along the b-direction is ≈7 Å, it follows that the Li⁺+H₂O and Na⁺+H₂O thicknesses are 2.5 Å and 2.0 Å, respectively.

FIG. 11b shows XRD patterns for powders that were first washed with LiCl solution then treated in HNO₃ or MgCl₂ aqueous solutions. The XRD patterns for the remaining cations are plotted in FIG. 11c. The initial d-spacing was ≈9.5 Å (top black pattern in FIGS. 11b and c) for Li⁺-intercalated NFs. When stirred in 0.1 M nitric acid, the 9.5 Å-peak slightly shifted to ≈9.3 Å (red pattern in FIG. 11b) suggesting the successful intercalation of hydronium ions between the NFs. Likewise, stirring the Li⁺-intercalated NFs in a 0.02 M MgCl₂ solution (green pattern in FIG. 11b), resulted in a d-spacing expansion from 9.5 Å to 10.8 Å which we take as evidence for the exchange of Li⁺ ions with Mg²⁺.

The situation is quite similar for other divalent cations (Mn²⁺, Fe²⁺, etc.). Cationic exchange between Li⁺, on one hand, and Mn²⁺, Fe²⁺, Co²⁺, Ni²⁺, and Zn²⁺, on the other hand, occurs when LiCl washed powders were further treated in 0.02 M aqueous solutions of the targeted cations. As shown in FIG. 11c, the resulting d-spacings after cationic exchange are 11.4 Å, 10.7 Å, 9.8 Å, 9.4 Å, and 8.9 Å for Mn²⁺, Fe²⁺, Co²⁺, Ni²⁺ and Zn²⁺-intercalated 1DL NFs, respectively.

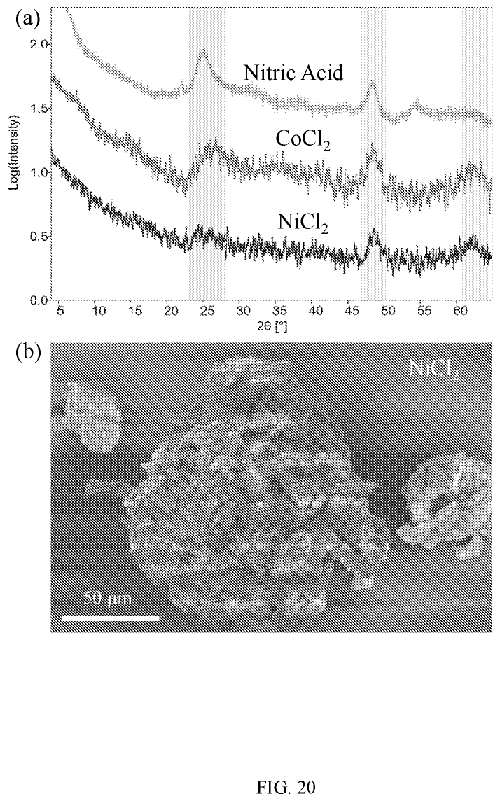

Interestingly, when EtOH washed powders were directly—i.e. without first exchanging the TMA⁺ with Li⁺-immersed in HNO₃, CoCl₂ or NiCl₂ aqueous solutions for 24 h or longer, the low angle peaks vanished (FIG. 20a) implying that any order along the b-stacking direction was destroyed. The only remaining peaks were the three non-basal reflections at 2θ values of ≈26°, ≈48° and ≈62° (red bands in FIG. 20a). A SEM micrograph of a NiCl₂-washed sample (FIG. 20b) confirms a change in the MPPs morphology into nanometer-sized, slightly porous agglomerates.

To summarize this section, the interfilamentous space between the 1DL NFs is quite readily exchangeable with monovalent (H₃O⁺, Li⁺, and Na⁺) and divalent cations (Mg²⁺, Mn²⁺, Fe²⁺, Co²⁺, Ni²⁺, and Zn²⁺). The corresponding XRD patterns clearly demonstrated the successful intercalation of these cations by the slight shifting of low angle (<10°) 2θ, peaks. One can note here that the non-basal peaks located at ˜48° and 62° 2θ (red dashed lines/bands in FIGS. 11a-c and 19a-c) perfectly lined up in all cases regardless to the nature of the intercalants. This result indirectly confirms the correctness of assigning these peaks to the 1DL backbone (see below).

### Surface Charge and Hydrodynamic Size of NFs Agglomerates

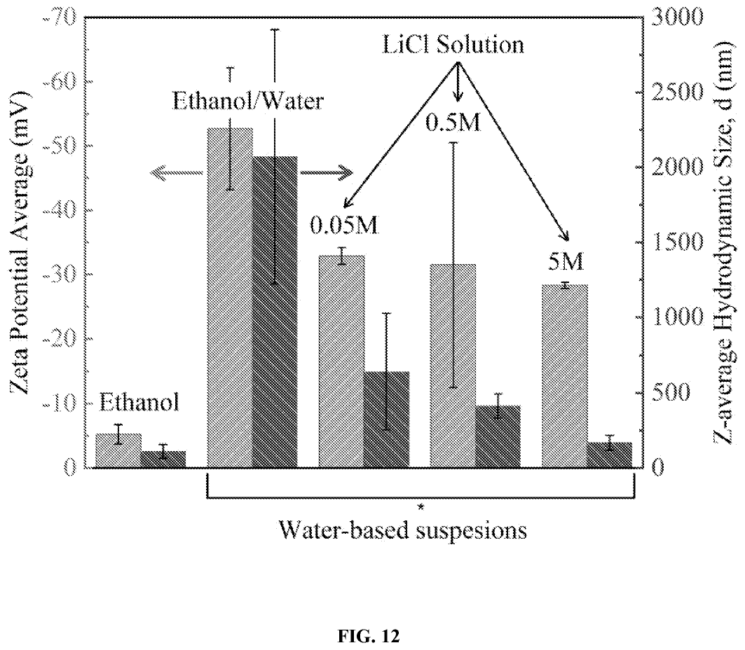

One objective of this work was to investigate the surface charge and aggregation behavior of the prepared NFs and NFs-based MPPs intercalated with TMA⁺ or Li⁺ cations. To that effect, we measured the zeta potentials, ζ, and hydrodynamic diameters, d_{H}, for powders, once after EtOH washing and another after washing with LiCl aqueous solutions. Note that apart from the first sample (top row in Table 1) that was washed and measured in EtOH, all other samples were washed with solvents/solution mentioned in first column in Table 1, then dispersed in DI water for ζ-potential and dH measurements. All measurements were repeated 3 times; results were averaged (see FIG. 12) and summarized in Table 1 below.

Table 1: Summary of ζ-potentials and Z-average hydrodynamic size, d_{H}, values measured as a function of washing media shown in left-hand column.

| | | Zeta Potential | | | |
|---|---|---|---|---|---|
| | | Average (mV) | | d_{H} (nm) | |
| Washing solution | | Average | Std. D | Average | Std. D |
| EtOH | | −5 | 1.5 | 111 | 45 |
| EtOH [symbol] H₂O | | −53 | 9.5 | 2071 | 850 |
| EtOH [symbol] | 0.05M LiCl | −33 | 1.3 | 640 | 390 |
| LiCl [symbol] H₂O | 0.5M LiCl | −31 | 19 | 414 | 80 |
| | 5.0M LiCl | −28 | 0.5 | 168 | 50 |

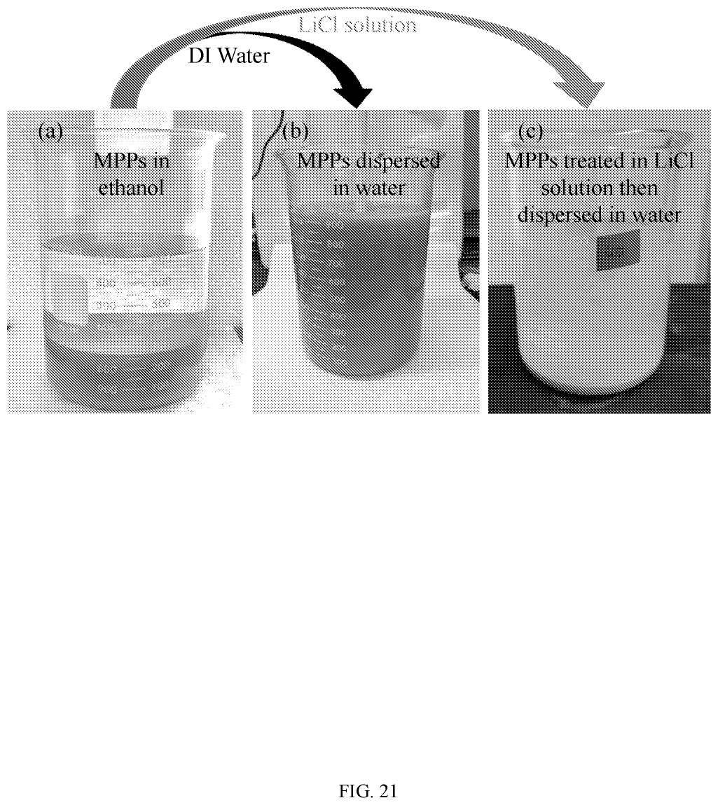

When the solvent was EtOH, the ζ-potential of the MPPs was −5±1.5 mV, which explains colloid instability in this solvent; the MPPs settled to the bottom of the container (FIG. 21a) and only particles/entities with dH of ˜0.1 μm remained suspended (right axis in FIG. 12). When the MPPs were dispersed in DI water, a ζ-potential of ˜−53±10 mV is recorded which, in turn, resulted in highly stable colloidal suspensions (see FIG. 21b). The high surface charge stabilized agglomerates of 1DL NFs as large as 2±0.8 μm (right axis in FIG. 12). The size of these aggregates is a function of colloidal concentration. Upon washing the MPPs—right after EtOH washing—with LiCl aqueous solutions, the ζ-potential values were reduced from −53±10 mV in DI water to −33±1.3 mV in 0.05 M LiCl solution (FIG. 12 and Table 1). As the molarity of the LiCl solution increased to 0.5 M and to 5 M, the ζ-potentials changed slightly to ≈−28±0.5 mV (FIG. 12 and Table 1 above).

When the MPPs (after washing with ethanol to neutral) were dried at 50° C. then redispersed in DI water there was a noticeable increase in the pH up to values of ˜10. That can be caused by the uptake of protons by O⁻ and/or OH⁻ surface terminations. In our case, however, we did not observed any changes in the low angle peaks suggesting that the surface oxygen atoms are hydroxylated through protonation.

To summarize, after neutralization in EtOH, the ζ-potential was slightly negative and most of MPPs settled (FIG. 21c). Only aggregates <1.4 μm in size were suspended in the EtOH (FIG. 12). Upon washing with water, at ≈−50±10 mV, the ζ-potential was quite negative which explains the colloidal stability in water.

The major drop in surface charge from DI water to 0.05M LiCl solution basically corresponds to Li⁺ ions electrostatic adsorption on the NFs negatively charged surfaces. Which in turn resulted in a decrease in the electrical double layer and a consequent drop in the repulsive electrostatic interaction between the NFs. The subsequent drop in surface charge as the molarity increased from 0.05 M to 5 M (FIG. 12 and Table 1) is further evidence for the decrease in the thickness of the diffuse layer of the 1DL NFs as the ionic strength increased.

### Thermal Stability of 1DL NFs

The thermal stabilities of our MPPs were explored using thermogravimetric analysis (TGA) in Ar up to 800° C. of NFs intercalated with TMA⁻, Li⁺ or Na⁺. All powders were dried at 50° C. in open air for 24 h prior to the TGA experiments. The TGA results were different for EtOH and salt washed samples. FIG. 23a. The latter lose weight until ≈250° C., before levelling off. The weight change at that temperature is predominantly due to loss of H₂O.²⁶ Washing with LiCl results in a slightly higher (18%) weight loss than washing with NaCl (15%) (FIG. 23a).

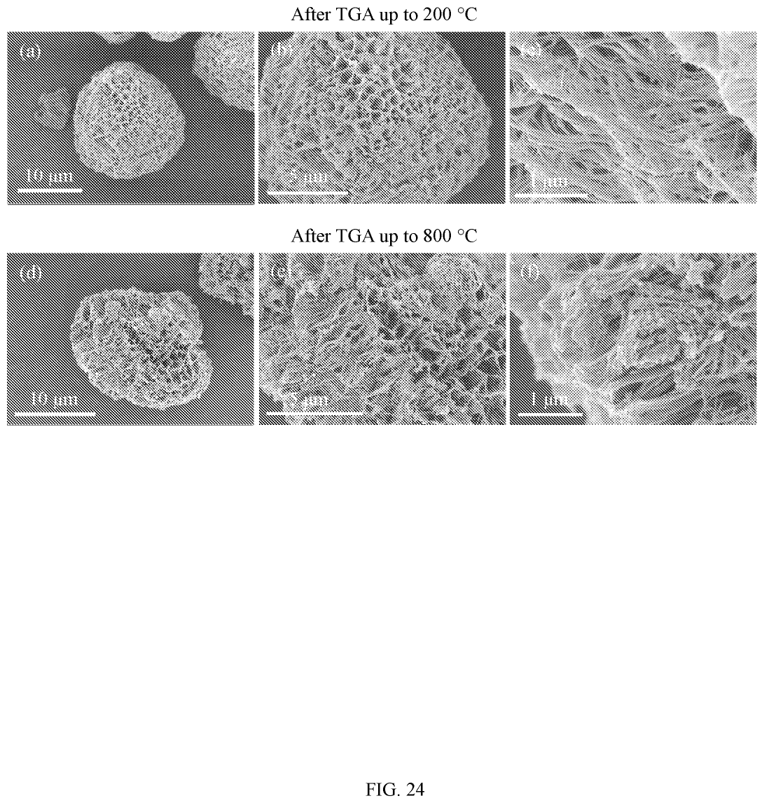

Heating EtOH-washed, TMA⁺-intercalated, NFs to 200° C., resulted in a ≈15% mass loss mostly probably due to residual EtOH solvent from washing (FIG. 23a). Upon further heating, at ≈350° C., there was another mass drop of ≈15% which is most probably due to the loss of hydration layers and intercalated TMA cations between the NFs (FIG. 23a). Heating to 200° C. does not change the XRD patterns (FIG. 23b). At higher magnifications (FIG. 24f) some of the NFs started spheroidizing/coarsening.

Heating the LiCl-washed MPPs led to one mass loss event of ≈17 wt. % up to ≈200° C. (FIG. 23a), that most likely corresponds to the loss of hydration layers associated with Li cations and/or dehydroxylation. The case for the NaCl-washed MPPs is quite similar, in that, the observed the mass loss, up to ≈200° C., was about 14 wt. % (FIG. 23a). The higher mass loss for the LiCl washed samples (17%) suggests that the number of water molecules associated with the Li ions are slightly higher than those of Na one, where weight loss is ≈15%. When the powders were further heated to 800° C., no further weight loss was observed.

XRD patterns, however, show that the Li⁺-intercalated NFs transformed to a mixture of rutile and lithium titanate, Li₂Ti₂O₄ (green pattern in FIG. 23c). After calcination at 800° C., the Na-intercalated NFs converted to a mixture of rutile and sodium titanate, Na₂Ti₆O₁₃.

### 1DL NFs Morphologies, Formation Mechanism and Self-Assembly

### Where the Reaction Occurs and Its Nature

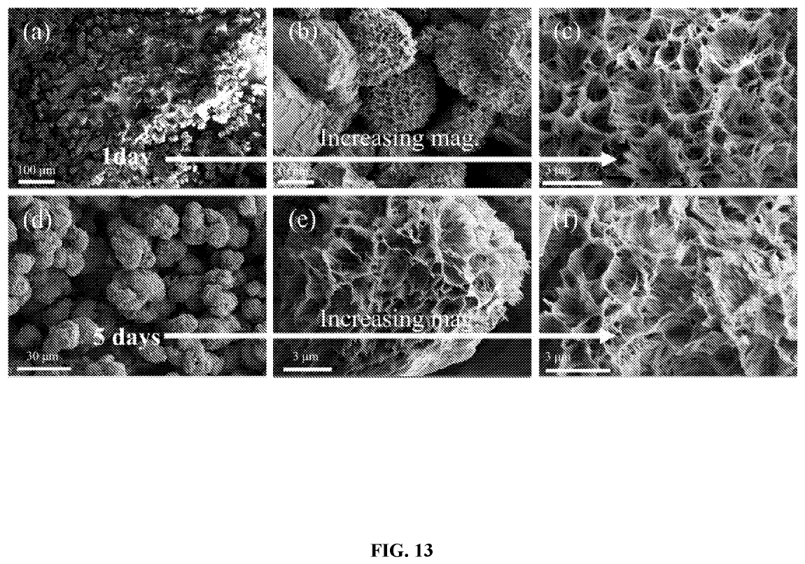

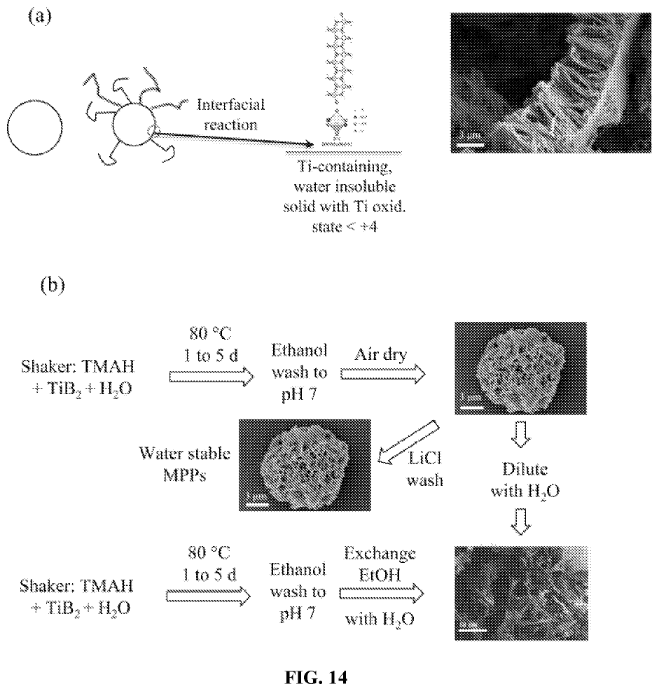

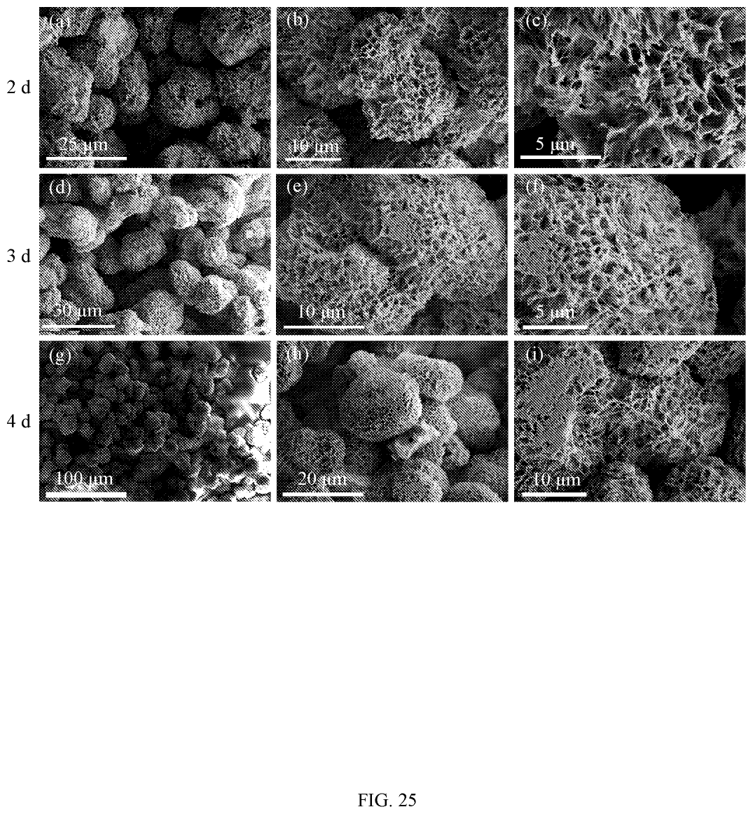

Herein, the XRD patterns of samples reacted for 1 to 5 d, shown in FIG. 9a make it clear that at early times strong TiB₂ peaks (dashed black lines in FIG. 9a) are present. The corresponding SEM micrographs (FIG. 13 and FIG. 25) clearly show that regardless of reaction times and at all magnifications, the MPPs surfaces appear to be identical. This is a useful observation because it indicates the TiB₂ to 1DL transformation starts at the surface and moves inwards into a central core with time. This also suggests that, at least initially, the reaction must be surface reaction rate controlled (see FIG. 26). FIG. 14a is a schematic of what we imagine is happening at the interface. First, Ti atoms are released into the reaction medium at which time they convert to TiO₆ octahedra. The latter then inserts itself between the receding substrate and the 1DL growing away from it. The implications of this conclusion are useful, because in principle it allows for the formation of core-shell configurations.

One of the major disadvantages of working with nanomaterials, in general, and nanoparticles in particular, is that after, typically, considerable effort goes into making them, they aggregate, and more processing is needed to disaggregate them, which in turn can introduce unwanted chemical and contaminants. It follows that the fact that our MPPs are free-flowing could be paradigm shifting in that we can now both have many of the advantages of reduced dimensions without their downside.

Lastly, the fact that the NFs nucleate on the surface of our precursors may also explain why these 1DL NFs have not been discovered earlier. In most sol-gel work to date, the starting point is a water-soluble Ti-source. We speculate that having the reaction nucleate on a solid surface allows the NFs to only grow in one dimension. More work is needed here. What is unmistakable, however, is that form of Ostwald-ripening is occurring in our microstructures. The primary particle size of our initial TiB₂ is in the 5 μm range, with few particles over 10 μm in size. As shown in inset in FIG. 9b, the final sizes of the MPPs are higher. This result not only confirms Ostwald-ripening as a mechanism, but demonstrates that our final product, 1DL NFs, can dissolve and re-principate in our reaction medium.

In summary, we report on a truly large-scale synthesis of TiO₂-based sub-nanostructures following a facile solution-precipitation method at ambient pressures and at temperatures of <100° C. Our method entails shaking water insoluble, cheap and commercial Ti-containing powder (for example, TiB₂) in TMAH aqueous solutions in plastic bottles at 80° C. for 1-5 days. The resulting powders, washed with EtOH and water using an overhead mixer and a beaker before they were let to dehydrate at 50° C. in open air. No centrifugation or filtration is needed in processing these nanomaterials which reduces their production cost at the industrial level. Low magnification SEM imaging revealed that each particle has sponge-like morphology with an average size of ˜13 μm. It has been further elaborated using HR-STEM and SAD patterns that the building unit of the MPPs are lepidocrocite-type titanate NFs that are 5×7 Å² in cross section and several microns long. According to out TGA results, the mesoporous morphology is stable up to 800° C.

We further investigated the capability of intercalating various cations in the interfilamentous gallery. Herein, we show that the NFs are readily ion exchangeable with various monovalent (H₃O⁺, Li⁺, and Na⁺) and divalent cations (Mg²⁺, Mn²⁺, Fe²⁺, Co²⁺, Ni²⁺ and Zn²⁺).

Lastly, we investigated the surface charge and hydrodynamic size of the self-assembled NFs and showed that the ζ-potentials can be >−60 mV in DI water resulting in a highly-stable colloidal suspensions.

### Experimental Procedures

### Materials Synthesis and Processing

### Sample Preparation of 1DL

Our scalable synthesis protocol entails mixing commercial TiB₂ (−325 mesh Thermo Scientific, PA, U.S.) powders with a TMAH aqueous solution (Alfa Aesar, 25 wt. % in DI water, 99.9999%) in polyethylene bottles. The Ti:TMAH mole ratio was kept constant at 0.6. In a typical batch, we immersed 100 g TiB₂ powder in 900 mL TMAH solution in 5 different polyethylene bottles, each 250 mL in volume. The bottles were then transferred to a temperature-controlled incubator/shaker (211DS 49L Shaking Incubator, Labnet International Inc., NC) and shook at 175 rpm at 80° C. for 1 to 5 d.

### Washing Protocol

After reaction, all the resulting sediment combined in a 1L beaker, and the powders were allowed to settle after which the supernatant was decanted and discarded. To wash away any unreacted TMAH, the 1 L beaker was again filled with EtOH (Decon Lab Inc., 200 proof), stirred at RT for 1 to 2 h using an overhead mixer (OSC-10L-200rpm, LabFish, China) after which the powders were again allowed to settle before the EtOH supernatant, comprised of excess TMA⁺ cations and other unwanted reaction products, was again dumped to waste. This procedure was repeated multiple times till the pH was ≈7, after which the powders were allowed to dry in open air at 50° C. overnight.

### Synthesis of Ions-Intercalated 1DL NFs

To assess capability of ion exchange, some powders, while wet, were further stirred on a stir plate, 3 subsequent times each of 6 h in one of the following salt solutions: LiCl 0.5M, LiCl 5M, NaCl 0.5M, or NaCl 5M and then rinsed with DI water 3 times to remove any unreacted salts and/or reaction products. All salts were purchased from Alfa Aesar with >99% purity. The LiCl- and NaCl-treated powders were then air-dried at 50° C. overnight.

### X-ray Diffraction, XRD

A diffractometer (Rigaku MiniFlex, Tokyo, Japan) operated with Cu Ka radiation (40 kV and 15 mA) was used to obtained XRD patterns. The powders were scanned in the 2-65° 2θ range with a step size of 0.02° and a dwell time of 1 s. Unless otherwise noted, all powders were dried overnight at 50° C. in open air before any XRD scans.

### Scanning Electron Microscopy

A scanning electron microscope, SEM (Zeiss Supra 50 VP, Carl Zeiss

SMT AG, Oberkochen, Germany) was used to obtain micrographs of our materials. The SEM settings were set to an in-lens detector, a 30 mm aperture, and an accelerating voltage of 3-5 kV.

### Particle Size Distribution

Particle size distribution was carried out by measuring both minimum and maximum lengths of each particle for a total of 100 particles using ImageJ software.

### Scanning Transmission Electron Microscopy

A scanning transmission electron microscope, STEM, using an aberration-corrected cold field emission JEOL ARM200CF operated at 200 kV primary electron energy. Imaging and spectroscopic measurements were conducted with the emission current at 15 μA, an electron probe semi-convergence angle of 24 mrad, as well as inner and outer detector angles of 68 mrad and 280 mrad for high angle annular dark field (HAADF) imaging. For low angle annular dark field (LAADF) imaging the inner and outer angles were 30 mrad and 120 mrad respectively. Annular bright field (ABF) imaging was conducted using an outer angle of 23 mrad and an inner angle of 11mrad.

To conduct nanoscale elemental identification and quantification, the ARM200CF is equipped with an Oxford XMX100TLE X-ray windowless silicon drift detector (SDD) with a 100 mm² detector area.

STEM samples were prepared by drop casting a 5 ml suspension of TiB₂-derived powders (5d, 80° C.) in mEtOH on a 3 mm lacey carbon copper grid. The sample was then allowed to dry for an hour before insertion into the microscope column.

### Electron Energy-loss Spectroscopy (EELS)

EELS measurements were conducted using a post-columns Gatan Continuum GIF ER spectrometer, with an electron probe semi-convergence angle of 17.8 mrad and a collection angle of 53.4 mrad. In situ cooling was conducted using a Gatan 636 liquid nitrogen, N₂, cold stage. To reduce the presence of latent water, the samples were heated to 100° C. for an hour inside the microscope column.

### Zetapotential and Particle Size Measurements

A Zetasizer (Nano-ZS, Malvern Panalytical, Malvern, U.K.) was used for the electrophoretic mobility measurements. The electrophoretic mobility values converted to zeta potentials, ¿, using the Smoluchowski model. The hydrodynamic diameter, dH, was also measured, on the same machine, using dynamic light scattering DLS. Average hydrodynamic diameter was calculated from the diffusion coefficient using the Strokes-Einstein equation. All measurements were carried out at ambient conditions with a holding equilibrium time of 120 s.

### Thermogravimetric Analysis, TGA

A thermobalance (TA Instruments Q50, New Castle, DE, USA) was used for the TGA analysis. Dry powders (˜40 mg) were loaded in sapphire crucible, heated at 10° C./min, under Ar flow at 10 mL/min, to 800° C., then system was let to cool down naturally.

### REFERENCES

1. Kumar, S.G., and Devi, L.G. (2011). Review on Modified TiO2 Photocatalysis under UV/Visible Light: Selected Results and Related Mechanisms on Interfacial Charge Carrier Transfer Dynamics. J. Phys. Chem. A 115, 13211-13241./10.1021/jp204364a.

2. Schaub, R., Thostrup, P., Lopez, N., Lægsgaard, E., Stensgaard, I., Nørskov, J.K., and Besenbacher, F. (2001). Oxygen vacancies as active sites for water dissociation on rutile TiO2 (110). PRL 87, 266104./10.1103/PhysRevLett.87.266104.

3. Wang, A.S.D., and Sasaki, T. (2014). Titanium Oxide Nanosheets: Graphene Analogues with Versatile Functionalities. Chem. Rev. 114, 9455-9486.

4. Bourikas, K., Kordulis, C., and Lycourghiotis, A. (2014). Titanium Dioxide (Anatase and Rutile): Surface Chemistry, Liquid-Solid Interface Chemistry, and Scientific Synthesis of Supported Catalysts. Chem. Rev. 114, 9754-9823./10.1021/cr300230q.

5. Wang, X., Li, Z., Shi, J., and Yu, Y. (2014). One-dimensional titanium dioxide nanomaterials: nanowires, nanorods, and nanobelts. Chem. Rev. 114, 9346-9384./10.1021/cr400633s.

6. Ramos-Delgado, N.A., Gracia-Pinilla, M.A., Mangalaraja, R. V., O'Shea, K., and Dionysiou, D.D. (2016). Industrial synthesis and characterization of nanophotocatalysts materials: titania. Nanotechnol. Rev. 5, 467-479. doi: 10.1515/ntrev-2016-0007.

7. Fang, W., Xing, M., and Zhang, J. (2017). Modifications on Reduced Titanium Dioxide Photocatalysts: A review. J. Photochem. Photobiolog C: Photochem. Rev. 32, 21-39./10.1016/j.jphotochemrev.2017.05.003.

8. Saeed, M., Muneer, M., and Akram, N. (2021). Photocatalysis: an effective tool for photodegradation of dyes-a review. Environ. Sci. Pollut. Res., 1-19./10.1007/s11356-021-16389-7.

9. Sasaki, T., Watanabe, M., Michiue, Y., Komatsu, Y., Izumi, F., and Takenouchi, S. (1995). Preparation and Acid-Base Properties of a Protonated Titanate with the Lepidocrocite-like Layer. Chem. Mater. 7, 1001.

10. Sasaki, T., and Watanabe, M. (1997). Semiconductor nanosheet crystallites of quasi-TiO2 and their optical properties. J. Phys. Chem. B. 101, 10159-10161./10.1021/jp9727658.

11. Zhou, W., Umezawa, N., Ma, R., Sakai, N., Ebina, Y., Sano, K., Liu, M., Ishida, Y., Aida, T., and Sasaki, T. (2018). Spontaneous Direct Band Gap, High Hole Mobility, and Huge Exciton Energy in Atomic-Thin TiO2 Nanosheet. Chem. Mater. 30, 6449-6457. 10.1021/acs.chemmater.8b02792.

12. Lai, Y., Lin, L., Pan, F., Huang, J., Song, R., Huang, Y., Lin, C., Fuchs, H., and Chi, L. (2013). Bioinspired Patterning with Extreme Wettability Contrast on TiO2 Nanotube Array Surface: A Versatile Platform for Biomedical Applications. Small 9, 2945-2953./10.1002/smll.201300187.

13. Hurum, D.C., Agrios, A.G., Gray, K.A., Rajh, T., and Thurnauer, M.C. (2003). Explaining the enhanced photocatalytic activity of Degussa P25 mixed-phase TiO2 using EPR. The Journal of Physical Chemistry B 107, 4545-4549.

14. Ohtani, B., Prieto-Mahaney, O., Li, D., and Abe, R. (2010). What is Degussa (Evonik) P25? Crystalline composition analysis, reconstruction from isolated pure particles and photocatalytic activity test. J. Photochemi. Photobiology A: Chemistry 216, 179-182.

15. Wang, X., Pehkonen, S.O., Rämö, J., Väänänen, M., Highfield, J.G., and Laasonen, K. (2012). Experimental and computational studies of nitrogen doped Degussa P25 TiO2: application to visible-light driven photo-oxidation of As (iii). Catalysis Science & Technology 2, 784-793.

16. Tobaldi, D.M., Pullar, R.C., Seabra, M.P., and Labrincha, J.A. (2014). Fully quantitative X-ray characterisation of Evonik Aeroxide TiO2 P25. Mater. Lett. 122, 345-347.

17. Sheng, L., Liao, T., Kou, L., and Sun, Z. (2017). Single-crystalline ultrathin 2D TiO2 nanosheets: A bridge towards superior photovoltaic devices. Mater. Today Energy 3, 32-39.10.1016/j.mtener.2016.12.004.

18. Esmat, M., Farghali, A.A., El-Dek, S.I., Khedr, M.H., Yamauchi, Y., Bando, Y., Fukata, N., and Ide, Y. (2019). Conversion of a 2D Lepidocrocite-Type Layered Titanate into Its 1D Nanowire Form with Enhancement of Cation Exchange and Photocatalytic Performance. Inorg. Chem. 58, 7989-7996./10.1021/acs.inorgchem.9b00722.

19. Badr, H., Natu, V., Neaţu, S., Neaţu, F., Kuncser, A., Rostas, A., Barsoum, M.W., and Florea, M. (2023). Ultra-stable, 1D TiO2 Lepidocrocite for Photocatalytic Hydrogen Production in Water-Methanol Mixtures. Matter. https://doi.org/10.1016/j.matt.2023.05.026.

20. Badr, H.O., Montazer, K., El-Melegy, T., Natu, V., Carey, M., Gawas, R., Phan, P., Qian Qian, Li, C.Y., Wiedwald, U., et al. (2022). Scalable, inexpensive, one-pot, facile synthesis of crystalline two-dimensional birnessite flakes. Matter 5, 2365-2381. https://doi.org/10.1016/j.matt.2022.05.038.

21. Kai., K., et al. (2008). Room-Temperature Synthesis of Manganese Oxide Monosheets. J. Am. Chem. Soc. 130, 15938-15943./10.1021/ja804503f.

22. Kai, K., Yoshida, Y., Kobayashi, Y., Kageyama, H., and Saito, G. (2012). Preparation and formation mechanism of a n-butylammonium/MnO2 layered hybrid via a one-pot synthesis under moderate conditions. Dalton Trans. 41, 825.

23. Zhang, A., Zhao, R., Hu, L., et al. (2021). Adjusting the Coordination Environment of Mn Enhances Supercapacitor Performance of MnO2. Adv. Energy Mater., 2101412./10.1002/aenm.202101412.

24. Zheng, W., Halim, J., Yang, L., Badr, H.O., Sun, Z., Persson, P.O.Å., Rosen, J., and Barsoum, M.W. (2022). MXene//MnO2 Asymmetric Supercapacitors with High Voltages and High Energy Densities. Batteries & Supercaps https://doi.org/10.1002/batt.202200151.

25. Sudhakar, K., Kono, T., El-Melegy, T., Badr, H.O., Laxmeesha, P.M., Montazeri, K., Semisalova, A., Farle, M., Wiedwald, U., and Barsoum, M.W. (2023). One pot, scalable synthesis of hydroxide derived ferrite magnetic nanoparticles. J. Magn. Magn. Mater. 582, 170986. https://doi.org/10.1016/j.jmmm.2023.170986.

26. Badr, H.O., El-Melegy, T., Carey, M., Natu, V., Hassig, M.Q., Johnson, C., Qian, Q., Li, C.Y., Kushnir, K., Ulloa, E.C., et al. (2022). Bottom-up, scalable synthesis of anatase nanofilament-based two-dimensional titanium carbo-oxide flakes. Mater. Today 54, 8-17. https://doi.org/10.1016/j.mattod.2021.10.033.

27. Badr, H.O., Lagunas, F., Autrey, D.E., Cope, J., Kono, T., Torita, T., Klie, R.F., Hu, Y.-J., and Barsoum, M.W. (2023). On the Structure of One-Dimensional TiO2 Lepidocrocite. Matter 6, 128-141. https://doi.org/10.1016/j.matt.2022.10.015.

28. Colin-Ulloa, E., Martin, J.L., Hanna, R.J., Frasch, M.H., Ramthun, R.R., Badr, H.O., Uzarski, J.R., Barsoum, M.W., Grimm, R.L., and Titova, L.V. (2023). Electronic Structure of 1D Lepidocrocite TiO2 as Revealed by Optical Absorption and Photoelectron Spectroscopy. J. Phys. Chem. C. 10.1021/acs.jpcc.2c06719.

29. Wang, L., Badr, H.O., Yanga, Y., Cope, J., Ma, E., Yuan, L., Li, Z., Barsoum, M.W., Chai, Z., and Shi, W. (2023). Unique Hierarchical Structures of One Dimensional Lepidocrocite Titanate with Cation-Exchangeable Sites for Extraordinary Selective Actinide Capture for Water Purification. JACS.

30. Wilson, O.R., Carey, M.S., Cope, J.H., Badr, H.O., Nantz, J.M., ElMelegy, T.A., Barsoum, M.W., and Magenau, A.J.D. (2023). Repairable reinforced composites of 1D TiO2 lepidocrocite mesoparticles and thiol-yne click networks via alkylborane-initiated in situ polymerization. Cell Rep. 4, 101434. https://doi.org/10.1016/j.xcrp.2023.101434.

31. Sudhakar, K., Karmakar, A., Badr, H., El-Melegy, T., Hassig, M.Q., Carey, M., Masiuk, S., Wu, L., Qian, Q., Kono, T., et al. (2023). One-dimensional, Lepidocrocite-based Nanofilaments and Their Self-Assembly. Matter. https://doi.org/10.1016/j.matt.2023.06.006.

32. Ma, R., and Sasaki, T. (2010). Nanosheets of Oxides and Hydroxides: Ultimate 2D Charge-Bearing Functional Crystallites. Adv. Mater. 22, 5082-5104./10.1002/adma.201001722.

33. Verger, L., Natu, V., Ghidiu, M., and Barsoum, M.W. (2019). Effect of Cationic Exchange on the Hydration and Swelling Behavior of Ti3C2Tz MXene. J. Phys. Chem. C 20044-20050, 19725-19733.

34. Varun Natu, Rahul Pai, Olivia Wilson, Edward Gadasu, Hussein Badr, Avishek Karmakar, Andrew J. D. Magenau, Vibha Kalra, a., and Barsoum+, M.W. (2022). Effect of Base/Nucleophile Treatment on Interlayer Ion Intercalation, Surface Terminations, and Osmotic Swelling of Ti3C2Tz MXene Multilayers. Chem. Mater. 34, 678-693.

35. Ma, R., Bando, Y., and Sasaki, T. (2003). Nanotubes of lepidocrocite titanates. Chem. Phys. Lett. 380, 577-582./10.1016/j.cplett.2003.09.069.

36. Sasaki, T., Watanabe, M., Hashizume, H., Yamada, H., and Nakazawa, H. (1996). Macromolecule-like Aspects for a Colloidal Suspension of an Exfoliated Titanate. Pairwise Association of Nanosheets and Dynamic Reassembling Process Initiated from It. J. Am. Chem. Soc. 118, 8329-8335./10.1021/ja960073b.

37. Yunxia Chen, Xin He, Xiujian Zhao, Qihua Yuan, and Gu, X. (2007). Preparation, characterization, and growth mechanism of a novel aligned nanosquare anatase in large quantities in the presence of TMAOH. J. Colloid Interface Sci. 310, 171-177. 10.1016/j.jcis.2007.01.046.

38. Tan, Z., Sato, K., and Ohara, S. (2015). Synthesis of layered nanostructured TiO2 by hydrothermal method. Adv Powder Technol 26, 296-302./10.1016/j.apt.2014.10.011.

39. Yuan, H., Besselink, R., Liao Z., and Elshof, J.E. (2014). The swelling transition of lepidocrocite-type protonated layered titanates into anatase under hydrothermal treatment. Sci. Rep. 4, 4584/10.1038/srep04584.

40. Liu, Z., Ooi, K., Kanoh, H., Tang, W., and Tomida, T. (2000). Swelling and Delamination Behaviors of Birnessite-Type Manganese Oxide by Intercalation of Tetraalkylammonium Ions. Langmuir 16, 4154-4164. 10.1021/la9913755.

41. Brock, S.L., et al. (1999). Particle Size Control and Self-Assembly Processes in Novel Colloids of Nanocrystalline Manganese Oxide. J. Phys. Chem. B 103, 7416-7428. 10.1021/jp991009u.

42. Sun, X., and Li, Y. (2003). Synthesis and Characterization of Ion-Exchangeable Titanate Nanotubes. Chem. Eur. J. 9, 2229-2238./10.1002/chem.200204394.

43. Mao, Y., and Wong, S.S. (2066). Size-and Shape-Dependent Transformation of Nanosized Titanate into Analogous Anatase Titania Nanostructures. J. AM. CHEM. SOC. 128, 8217-8226./10.1021/ja0607483.

44. Liua, W., Sun, W., Borthwick, A.G.L., and Ni, J. (2013). Comparison on aggregation and sedimentation of titanium dioxide, titanate nanotubes and titanate nanotubes-TiO2: Influence of pH, ionic strength and natural organic matter. Colloids Surf, A Physicochem Eng Asp 434, 319-328./10.1016/j.colsurfa.2013.05.010.

45. Bavykin, D.V., Friedrich, J.M., and Walsh, F.C. (2006). Protonated Titanates and TiO2 Nanostructured Materials: Synthesis, Properties, and Applications. Adv. Mater. 18, 2807-2824./10.1002/adma.200502696.

46. Yang, J., Jin, Z., Wang, X., Li, W., Zhang, J., Zhang, S., Guo, X., and Zhang, Z. (2003). Study on composition, structure and formation process of nanotube Na2Ti204 (OH) 2. Dalton Trans., 3898-3901./10.1039/B305585J.

47. Phillips, P.J., Paulauskas, T., Rowlands, N., Nicholls, A. W., Low, K.B., Bhadare, S., and Klie, R.F. (2014). A New Silicon Drift Detector for High Spatial Resolution STEM-XEDS: Performance and Applications. Microsc. Microanal. 20, 1046-1052.10.1017/S1431927614001639.

### Aspects

The following Aspects are illustrative only and do not limit the scope of the present disclosure or the appended claims. Any one or more parts of any one or more Aspects can be combined with any one or more parts of any one or more other Aspects.

Aspect 1. A composition, comprising: a plurality of metal oxide subnanofilaments and/or nanofilaments, the subnanofilaments and/or nanofilaments optionally comprising a lepidocrocitic region, the plurality of metal oxide subnanofilaments and/or nanofilaments optionally comprising an amount of carbon, the plurality of metal oxide subnanofilaments and/or nanofilaments optionally being comprised in a bundle, in a flake, or in both a flake and a bundle.

Aspect 2. The composition of claim 1, wherein at least some of the nanofilaments and/or subnanofilaments have a width in the range of from about 3 to about 50 Å, for example, from about 7 to about 20 Å.

Aspect 3. The composition of Aspect 1, wherein at least some of the nanofilaments and/or subnanofilaments include Ti atoms. The Tia atoms can be, for example, arranged in a zig zag nature.

Aspect 4. The composition of Aspect 1, wherein the nanofilaments and/or subnanofilaments define a non-circular cross-section. Such a cross-section can be, for example, oval.

Aspect 5. The composition of Aspect 4, wherein the nanofilaments and/or subnanofilaments define a cross-sectional aspect ratio of from greater than 1 to about 10.

Aspect 6. The composition of Aspect 5, wherein the nanofilaments and/or subnanofilaments define a cross-sectional aspect ratio of from about 2 to about 5.

Aspect 7. The composition of Aspect 1, wherein the nanofilaments and/or subnanofilaments have an average cross-sectional area in the range of from about 10 to about 100 Å².

Aspect 8. The composition of Aspect 1, wherein at least some of the nanofilaments and/or subnanofilaments have a length in the range of from 1 nm to about 25 μm.

Aspect 9. The composition of Aspect 8, wherein at least some of the nanofilaments and/or subnanofilaments have a length in the range of from 1 nm to about 1 μm.

Aspect 10. The composition of Aspect 1, wherein the nanofilaments and/or subnanofilaments are comprised in a plurality of flakes.

Aspect 11. The composition of Aspect 1, wherein at least some of the plurality of the nanofilaments and/or subnanofilaments lie in a common plane.

Aspect 12. The composition of any one of Aspects 1-11, further comprising a pharmaceutically acceptable carrier.

Aspect 13. The composition of Aspect 1, further comprising a binder.

Aspect 14. The composition of Aspect 13, wherein the binder comprises a polymer.

Aspect 15. A device, the device comprising a composition according to Aspect 1.

Aspect 16. The device of Aspect 15, wherein the device is characterized as an energy storage device.

Aspect 17. The device of Aspect 15, wherein the device comprises an electrode.

Aspect 18. The device of Aspect 17, wherein the electrode comprises the composition according to Aspect 1.

Aspect 19. The device of Aspect 15, wherein the device comprises a dispenser, the dispenser having disposed therein the composition according to Aspect 1.

Aspect 20. A method, comprising: contacting a mono-, binary, ternary, or higher carbide, nitride, boride, phosphide, aluminide, or silicide, or titanium metal with a quaternary ammonium salt and/or base, the mono-, binary, ternary, or higher carbide, nitride, boride, phosphide, aluminide, or silicide, or titanium metal optionally being non-water-soluble, the non-water soluble binary, or ternary, or higher carbide, nitride, boride, phosphide, aluminide, or silicide optionally comprising a transition metal, the transition metal optionally comprising titanium, the contacting being performed under conditions sufficient to give rise to a nanofilamentous product.

Aspect 21. The method of Aspect 20, wherein the conditions comprise a temperature of from 0 to 100° C. for from about 5 hours to about 1 week.

Aspect 22. The method of Aspect 20, comprising contacting a binary, ternary, or higher boride with a quaternary ammonium salt and/or base so as to give rise to a nanofilamentous product.

Aspect 23. The method of Aspect 22, wherein the binary boride comprises one or more titanium borides.

Aspect 24. The method of Aspect 22, wherein the quaternary ammonium salt and/or base comprises an ammonium hydroxide, an ammonium halide, or any combination thereof.

Aspect 25. The method of Aspect 24, wherein the ammonium hydroxide comprises tetramethylammonium hydroxide (TMAOH), tetraethylammonium hydroxide (TEAOH), tetrapropylammonium hydroxide (TPAOH), tetrabutylammonium hydroxide (TBAOH), ammonium hydroxide (NH₄OH), their amine derivatives, or any combination thereof.

Aspect 26. The method of Aspect 24, wherein the quaternary ammonium salt comprises a quaternary ammonium chloride, a quaternary ammonium bromide, a quaternary ammonium iodide, a quaternary ammonium fluoride, or any combination thereof.

Aspect 27. The method of Aspect 20, further comprising filtering the product.

Aspect 28. The method of Aspect 20, further comprising washing the product with a metal salt and/or other water-soluble metal compounds.

Aspect 29. The method of Aspect 20, further comprising washing the product with a metal salt and/or water-soluble metal compounds, the metal salt optionally comprising metal sulfate, nitrate, chromate, acetate, carbonate, permanganate, or metal hydroxide, or any combination of thereof.

Aspect 30. The method of Aspect 29, wherein a metal in the metal salt comprises Li, Na, K, Cs, Mg, Ca, Cr, Mn, Fe, Co, Ni, Cu, Zn, Nb, Mo, Cd, Ta, or W, or any combination of thereof.

Aspect 31. The method of Aspect 29, wherein the metal salt comprises LiCl, KCl, NaCl, CsCl, LiF, KF, NaF, LiOH, KOH, NaOH, or any combination thereof.

Aspect 32. The method of Aspect 29, wherein the metal salt comprises CrCl₃, MnCl₂, FeCl₂, FeCl₃, CoCl₂, NiCl₂, MoCl₅, FeSO₄, (NH₄)₂Fe(SO₄)₂, CuCl₂, CuCl, ZnCl₂ or any combination thereof.

Aspect 33. The method of Aspect 20, wherein the product is a composition according to Aspect 1.

Aspect 34. A method, comprising: contacting particulate TiO₂ with a quaternary ammonium salt and/or base, the contacting being performed under conditions sufficient to give rise to a nanoparticulate product, the nanoparticulate product optionally at least some nanoparticles having a diameter of from about 2 nm to about 1000 nm, optionally from about 10 to about 100 nm.

Aspect 35. The method of Aspect 34, wherein the quaternary ammonium salt and/or base comprise an ammonium hydroxide, an ammonium halide, or any combination thereof.

Aspect 36. The method of Aspect 34, wherein the quaternary ammonium base comprises tetramethylammonium hydroxide (TMAOH), tetraethylammonium hydroxide (TEAOH), tetrapropylammonium hydroxide (TPAOH), tetrabutylammonium hydroxide (TBAOH), ammonium hydroxide (NH4OH), their amine derivatives, or any combination thereof.

Aspect 37. The method of Aspect 34, wherein the quaternary ammonium salt comprises a quaternary ammonium chloride, a quaternary ammonium bromide, a quaternary ammonium iodide, a quaternary ammonium fluoride, or any combination thereof together with a base.

Aspect 38. The method of Aspect 34, further comprising filtering the product.

Aspect 39. A composition, comprising a population of nanoparticles made according to Aspect 34.

Aspect 40. A method, comprising replacing TiO₂ with a population of nanoparticles made according to Aspect 34.

Aspect 41. A method, comprising: contacting a mono-, binary, ternary, or higher carbide, nitride, boride, phosphide, aluminide, or silicide, or titanium metal with a quaternary ammonium salt and/or base, the mono-, binary, ternary, or higher carbide, nitride, boride, phosphide, aluminide, or silicide, or titanium metal optionally being non-water-soluble, the non-water soluble binary, or ternary, or higher carbide, nitride, boride, phosphide, aluminide, or silicide optionally comprising a transition metal, the transition metal optionally comprising titanium, the contacting optionally performed while shaking, and the contacting being performed under conditions sufficient to give rise to mesoporous particles.

Aspect 42. A method, comprising: contacting a mono-, binary, ternary, or higher carbide, nitride, boride, phosphide, aluminide, or silicide, or titanium metal with a quaternary ammonium salt and/or base, the mono-, binary, ternary, or higher carbide, nitride, boride, phosphide, aluminide, or silicide, or titanium metal optionally being non-water-soluble, the non-water soluble binary, or ternary, or higher carbide, nitride, boride, phosphide, aluminide, or silicide optionally comprising a transition metal, the transition metal optionally comprising titanium, the contacting optionally performed while shaking, the contacting being followed by washing with at least one salt and performed under conditions sufficient to give rise to mesoporous particles.

Aspect 43. A method, comprising: contacting a mono-, binary, ternary, or higher carbide, nitride, boride, phosphide, aluminide, or silicide, or titanium metal with a quaternary ammonium salt and/or base, the mono-, binary, ternary, or higher carbide, nitride, boride, phosphide, aluminide, or silicide, or titanium metal optionally being non-water-soluble, the non-water soluble binary, or ternary, or higher carbide, nitride, boride, phosphide, aluminide, or silicide optionally comprising a transition metal, the transition metal optionally comprising titanium, the contacting performed while shaking and at a temperature of from about 50 to about 95° C., followed by washing with LiCl to give rise to mesoporous particles.

Aspect 44. A composition comprising mesoporous particles made according to any one of Aspects 41-43.

Aspect 45. A composition according to Aspect 44, further comprising a therapeutic.

Aspect 46. An electrode, the electrode comprising a composition according to Aspect 44.

Aspect 47. A device, the device comprising a composition according to Aspect 44.

Aspect 48. The device of Aspect 47, wherein the device is an energy storage device.

Aspect 49. A method, the method comprising operating the device of Aspect 47.

Aspect 50. A mesoporous particle, comprising: a plurality of lepidocrocitic nanofilaments, the plurality of lepidocrocitic nanofilaments optionally comprising Ti, the mesoporous particle having a diameter in the range of from about 1 to about 30 μm.

Aspect 51. The mesoporous particle of Aspect 50, wherein the mesoporous particle has a diameter of from about 2 to about 25 μm.

Aspect 52. The mesoporous particle of Aspect 50, wherein a nanofilament comprises a plurality of Ti atoms arranged in a zig-zag nature.

Aspect 53. A composition, comprising a plurality of mesoporous particles according to Aspect 50.

Aspect 54. A colloid, the colloid comprising a plurality of mesoporous particles according to Aspect 50.

Aspect 55. The colloid of Aspect 54, wherein the plurality of mesoporous particles is suspended in water.

Aspect 56. The colloid of Aspect 54, wherein the plurality of mesoporous particles is suspended in an aqueous medium.

1. A composition, comprising:
a plurality of metal oxide subnanofilaments and/or nanofilaments,
the subnanofilaments and/or nanofilaments optionally comprising a lepidocrocitic region,
the plurality of metal oxide subnanofilaments and/or nanofilaments optionally comprising an amount of carbon,
the plurality of metal oxide subnanofilaments and/or nanofilaments optionally being comprised in a bundle, in a flake, or in both a flake and a bundle.

2. The composition of claim 1, wherein at least one of (i) at least some of the nanofilaments and/or subnanofilaments have a width in the range of from about 3 to about 50 Å, (ii) at least some of the nanofilaments and/or subnanofilaments comprise Ti atoms, (iii) the nanofilaments and/or subnanofilaments define a non-circular cross-section.

3. (canceled)

4. (canceled)

5. The composition of claim 24, wherein the nanofilaments and/or subnanofilaments define a non-circular cross-section and wherein the nanofilaments and/or subnanofilaments define a cross-sectional aspect ratio of from greater than 1 to about 10.

6. (canceled)

7. The composition of claim 1, wherein the nanofilaments and/or subnanofilaments have an average cross-sectional area in the range of from about 10 to about 100 A².

8. The composition of claim 1, wherein at least some of the nanofilaments and/or subnanofilaments have a length in the range of from 1 nm to about 25 μm, optionally in the range of from 1 nm to about 1 μm.

9. (canceled)

10. The composition of claim 1, wherein the nanofilaments and/or subnanofilaments are comprised in a plurality of flakes.

11. (canceled)

12. The composition of claim 1, further comprising a pharmaceutically acceptable carrier.

13. The composition of claim 1, further comprising a binder, the binder optionally comprising a polymer.

14. (canceled)

15. A device, the device comprising a composition according to claim 1.

16. The device of claim 15, wherein the device is characterized as an energy storage device.

17. The device of claim 15, wherein the device comprises an electrode, the electrode optionally comprising the composition according to claim 1.

18. (canceled)

19. (canceled)

20. A method, comprising:
contacting a mono-, binary, ternary, or higher carbide, nitride, boride, phosphide, aluminide, or silicide, or titanium metal with a quaternary ammonium salt and/or base,
the mono-, binary, ternary, or higher carbide, nitride, boride, phosphide, aluminide, or silicide, or titanium metal optionally being non-water-soluble,
the non-water soluble binary, or ternary, or higher carbide, nitride, boride, phosphide, aluminide, or silicide optionally comprising a transition metal, the transition metal optionally comprising titanium,
the contacting being performed under conditions sufficient to give rise to a nanofilamentous product.

21. The method of claim 20, wherein the conditions comprise a temperature of from 0 to 100° C. for from about 5 hours to about 1 week.

22. The method of claim 20, comprising contacting a binary, ternary, or higher boride with a quaternary ammonium salt and/or base so as to give rise to a nanofilamentous product.

23. The method of claim 22, wherein the binary boride comprises one or more titanium borides.

24. The method of claim 22, wherein the quaternary ammonium salt and/or base comprises an ammonium hydroxide, an ammonium halide, or any combination thereof.

25. The method of claim 24, wherein the ammonium hydroxide comprises tetramethylammonium hydroxide (TMAOH), tetraethylammonium hydroxide (TEAOH), tetrapropylammonium hydroxide (TPAOH), tetrabutylammonium hydroxide (TBAOH), ammonium hydroxide (NH₄OH), their amine derivatives, or any combination thereof.

26. The method of claim 24, wherein the quaternary ammonium salt comprises a quaternary ammonium chloride, a quaternary ammonium bromide, a quaternary ammonium iodide, a quaternary ammonium fluoride, or any combination thereof.

27. (canceled)

28. The method of claim 20, further comprising washing the product with a metal salt and/or other water-soluble metal compounds.

29. The method of claim 20, further comprising washing the product with a metal salt and/or water-soluble metal compounds, the metal salt optionally comprising metal sulfate, nitrate, chromate, acetate, carbonate, permanganate, or metal hydroxide, or any combination of thereof.

30. The method of claim 29, wherein a metal in the metal salt comprises Li, Na, K, Cs, Mg, Ca, Cr, Mn, Fe, Co, Ni, Cu, Zn, Nb, Mo, Cd, Ta, or W, or any combination of thereof.

31. The method of claim 29, wherein the metal salt comprises LiCl, KCl, NaCl, CsCl, LiF, KF, NaF, LiOH, KOH, NaOH, or any combination thereof.

32. The method of claim 29, wherein the metal salt comprises CrCl₃, MnCl₂, FeCl₂, FeCl₃, CoCl₂, NiCl₂, MoCl₅, FeSO₄, (NH₄)₂Fe(SO₄)₂, CuCl₂, CuCl, ZnCl₂ or any combination thereof.

33. The method of claim 20, wherein the product is a composition according to claim 1.

34. A method, comprising:
contacting particulate TiO₂ with a quaternary ammonium salt and/or base,
the contacting being performed under conditions sufficient to give rise to a nanoparticulate product,
the nanoparticulate product optionally at least some nanoparticles having a diameter of from about 2 nm to about 1000 nm, optionally from about 10 to about 100 nm.

35. The method of claim 34, wherein the quaternary ammonium salt and/or base comprise an ammonium hydroxide, an ammonium halide, or any combination thereof.

36. The method of claim 34, wherein the quaternary ammonium base comprises tetramethylammonium hydroxide (TMAOH), tetraethylammonium hydroxide (TEAOH), tetrapropylammonium hydroxide (TPAOH), tetrabutylammonium hydroxide (TBAOH), ammonium hydroxide (NH₄OH), their amine derivatives, or any combination thereof.

37. The method of claim 34, wherein the quaternary ammonium salt comprises a quaternary ammonium chloride, a quaternary ammonium bromide, a quaternary ammonium iodide, a quaternary ammonium fluoride, or any combination thereof together with a base.

38. (canceled)

39. A composition, comprising a population of nanoparticles made according to claim 34.

40. A method, comprising replacing TiO₂ with a population of nanoparticles made according to claim 34.

41. A method, comprising:
contacting a mono-, binary, ternary, or higher carbide, nitride, boride, phosphide, aluminide, or silicide, or titanium metal with a quaternary ammonium salt and/or base,
the mono-, binary, ternary, or higher carbide, nitride, boride, phosphide, aluminide, or silicide, or titanium metal optionally being non-water-soluble,
the non-water soluble binary, or ternary, or higher carbide, nitride, boride, phosphide, aluminide, or silicide optionally comprising a transition metal, the transition metal optionally comprising titanium,
the contacting optionally performed while shaking, and
the contacting being performed under conditions sufficient to give rise to mesoporous particles.

42. A method, comprising:
contacting a mono-, binary, ternary, or higher carbide, nitride, boride, phosphide, aluminide, or silicide, or titanium metal with a quaternary ammonium salt and/or base, the mono-, binary, ternary, or higher carbide, nitride, boride, phosphide, aluminide, or silicide, or titanium metal optionally being non-water-soluble,
the non-water soluble binary, or ternary, or higher carbide, nitride, boride, phosphide, aluminide, or silicide optionally comprising a transition metal, the transition metal optionally comprising titanium,
the contacting optionally performed while shaking, the contacting being followed by washing with at least one salt and performed under conditions sufficient to give rise to mesoporous particles.

43. A method, comprising:
contacting a mono-, binary, ternary, or higher carbide, nitride, boride, phosphide, aluminide, or silicide, or titanium metal with a quaternary ammonium salt and/or base, the mono-, binary, ternary, or higher carbide, nitride, boride, phosphide, aluminide, or silicide, or titanium metal optionally being non-water-soluble,
the non-water soluble binary, or ternary, or higher carbide, nitride, boride, phosphide, aluminide, or silicide optionally comprising a transition metal, the transition metal optionally comprising titanium,
the contacting performed while shaking and at a temperature of from about 50 to about 95° C., followed by washing with LiCl to give rise to mesoporous particles.

44. (canceled)

45. (canceled)

46. (canceled)

47. (canceled)

48. (canceled)

49. (canceled)

50. A mesoporous particle, comprising:
a plurality of lepidocrocitic nanofilaments,
the plurality of lepidocrocitic nanofilaments optionally comprising Ti,
the mesoporous particle having a diameter in the range of from about 1 to about 30 μm.

51. (canceled)

52. The mesoporous particle of claim 50, wherein a nanofilament comprises a plurality of Ti atoms arranged in a zig-zag nature.

53. (canceled)

54. A colloid, the colloid comprising a plurality of mesoporous particles according to claim 50.

55. (canceled)

56. (canceled)
